(19) United States
(12) Patent Application Publication
(10) Pub. No.: US 2012/0251609 A1
(43) Pub. Date: Oct. 4, 2012

(54) DEMINERALIZED CANCELLOUS BONE MATRIX

(76) Inventors: Yen-Chen Huang, East Brunswick, NJ (US); Moon Hae Sunwoo, Old Tappan, NJ (US); Arthur A. Gertzman, Flemington, NJ (US)

(21) Appl. No.: 13/074,571

(22) Filed: Mar. 29, 2011

(51) Int. Cl. A61K 35/14; A61P 19/08; A61F 2/28; A61K 35/16; A61K 35/32

(52) U.S. Cl. 424/423; 424/93.7; 424/529; 424/530; 424/549; 623/23.63

(57) ABSTRACT

A demineralized cancellous bone matrix comprising a cancellous bone matrix that has been demineralized is described herein. The demineralized cancellous bone matrix is rigid and has certain dimensions, including a certain length. Implants comprising at least one demineralized cancellous bone matrix are also described. Also disclosed are methods for treating bone having a void or defect in a patient using at least one demineralized cancellous bone matrix. In addition, methods of making a demineralized cancellous bone matrix are disclosed.

### FIELD OF THE INVENTION

A demineralized cancellous bone matrix comprising a cancellous bone matrix that has been demineralized is described herein. The demineralized cancellous bone matrix is rigid and has certain dimensions, including a certain length. Implants comprising at least one demineralized cancellous bone matrix are also described. Also disclosed are methods for treating bone having a void or defect in a patient using at least one demineralized cancellous bone matrix. In addition, methods of making a demineralized cancellous bone matrix are disclosed.

### BACKGROUND

Bone voids or defects can be caused by various factors, including for example bone trauma, fractures due to an accident or other incident, bone diseases, bone disorders, bone tumors (e.g., cancer), bone loss, surgery, infections, metabolic disorders of bone and soft tissue, or developmental malformations. Bone voids and defects can affect bone strength, function and/or integrity. Bones of the skull, face, spine, hips, legs and feet, as well as other bones, may be affected by bone voids or defects. Bone repair procedures include, for instance, filling of bone voids and defects. Bone repair procedures can also include, without limitation, fracture repair, postero-lateral spinal fusion, interbody spine fusion and bone cyst filling.

With respect to fractures, these can occur in various bones. For example, fractures can occur in long bones, such as those in the arms and legs; hip bones; vertebra; cranium; as well as bones in the hands and feet. The fractures can result from trauma, such as a fall or a vehicular accident or other causes. Also, fractures of the bones can be stress fractures that result from repeated exposure of the bones to forces.

One type of fracture that is common in the elderly who suffer from osteoporosis is the compression or burst fracture in the vertebral bodies of the spine. There are approximately 700,000 cases of pathologic vertebral body compression fractures reported annually in the United States. As the patient's bone weakens, the vertebral bodies lose height and collapse, leading to severe pain and deformity. Burst and compression fractures of the vertebral bodies also occur in trauma cases, again leading to pain and deformities.

Bone void fillers can be natural or synthetic materials that are placed into a bone defect, such as a fracture, during a surgical procedure to assist in bone regeneration. They fill a bone void or bridge a gap between bone segments. They also may provide a three-dimensional scaffold for bone to grow on. Bone may be used as a bone void filler, such as bone from autograft (acquired from the same individual that will receive the implant), allograft (other human sources, usually cadavers) or xenograft (transplant from another species) sources. In addition, bone filler materials also include bone graft substitutes, such as collagen, polymers (e.g., silicone and some acrylics), hydroxyapatite, calcium sulfate and ceramics.

Bone cement (e.g., polymethylmethacylate (“PMMA”) is often used as a bone void filler to treat bone voids or defects. For example, it can be used to repair fractured bones, such as a leg bone. Also, it can be used to treat fractures in vertebral bodies. Bone cement (e.g., PMMA) is often used either in procedures that involve direct injection of the bone cement into the fractured vertebral body (i.e., vertebroplasty) or injection of the bone cement into the vertebral body after the height of the vertebral body is restored using a pressurized balloon (i.e., kyphoplasty). One of the disadvantages of using bone cement is that, once it is injected inside the patient, the bone cement is an inorganic material that acts as a foreign body, and thus, does not allow for complete healing and may instead lead to bone disease. Moreover, bone cement is typically stiffer than bone, which may increase the incidence of adjacent level fractures in the spine. Also, bone cement leakage may cause complications, and has been reported to occur in vetebroplasty and kyphoplasty procedures. If leakage does occur, PMMA bone cements can cause soft tissue injury due to the high temperatures of the exothermic polymerization reaction. In addition, PMMA forced into the vascular system can cause emboli.

In addition to spinal fractures, another common spinal condition is mild to severe degenerative disc disease. A healthy intervertebral disc facilitates motion between pairs of vertebrae while adsorbing and distributing compression forces and torque forces. The disc is composed of two parts; namely a tough outer ring (the annulus fibrosis (AF)), which holds and stabilizes a soft central core material (the nucleus pulposus (NP)) that bears the majority of the load forces. With degenerative disc disease, the onset of the degenerative cascade in the intervertebral disc(s) is typically associated with dehydration and loss of volume of the NP. The NP may then leak or bulge into the AF, and either or both of the NP and AF may come into contact with spinal nerves. This can cause inflammation or micromotion instability, resulting in pain, and loss of motion.

When an intervertebral disc is deformed, ruptured, diseased, or degenerating, surgical treatment can consist of augmenting or repairing the disc. For example, materials may be implanted or injected into the disc to replace or augment the NP. Also, to treat intervertebral discs, surgical treatments have been used to create a fusion between the two adjacent vertebral bodies. Prior approaches to vertebral fusion have involved substantial invasive surgery. It would be advantageous to have a vertebral fusion using an implant that is minimally invasive. In order to achieve a successful minimally invasive delivery of the implant into the disc space for fusion, the implant material should be able to easily pass through a small diameter cannula into the surgical site without jamming or wedging.

Moreover, it is desirable to have a maximum amount of surface area onto which new bone can begin to grow or form. In addition, certain materials that have been used do not allow bone healing through the entire implant to achieve complete interbody fusion since they may be synthetic materials that do not remodel into bone. Additionally, while implants have been used for spinal fusion, it has not always been possible to size an implant to fit the implant site. Also, the implants have not necessarily had the ability to conform to the shape of the implant site such that the contact between the implant and the endplates of the vertebral bodies is maximized. Moreover, the materials that have been used for vertebral fusion, such as titanium and polyether-etherketone (PEEK), do not always provide the optimal degree of mechanical support. Also, implant materials that are radiopaque do not allow for newly formed bone to be readily detected during follow-up x-rays.

Accordingly, there is a need for approaches, particularly minimally invasive approaches, to repair bone voids or defects. Also a bone material for repairing bone voids or defects that avoid leaking, and allows for easy handling and delivery, as well as complete healing post-implantation, is also desirable. Therefore, minimally invasive approaches, in which the implant materials do not jam or wedge during delivery from tubes or containers to implant sites, are desirable. Also desirable are implant materials that promote bone growth or healing, can be sized and can conform to the shape of the implant site, provide adequate mechanical support/load bearing and/or are at least partially radiolucent.

### SUMMARY OF THE INVENTION

Described herein are a demineralized cancellous bone matrix and implants comprising at least one demineralized cancellous bone matrix. The demineralized cancellous bone matrix comprises a cancellous bone matrix that has been demineralized. In certain embodiments, the cancellous bone matrix has been demineralized to contain about 5% to about 20% by weight residual calcium, or about 6% to about 15% by weight residual calcium, or about 8% to about 12% by weight residual calcium. In some embodiments, the implant comprises one, two, three, four or more demineralized cancellous bone matrices. In certain embodiments, at least 95%, 90% or 80% or the majority of the demineralized cancellous bone matrices of an implant contain about 5% to about 20% by weight residual calcium, or about 6% to about 15% by weight residual calcium, or about 8% to about 12% by weight residual calcium.

Furthermore, in some embodiments, the demineralized cancellous bone matrix is rigid. Also, in some embodiments, the demineralized cancellous bone matrix has dimensions, including a length, of between 0.5 mm to about 20 mm. In other embodiments, the demineralized cancellous bone matrix can have dimensions, including a length, of between about 2 mm to about 12 mm or between about 5 mm to about 10 mm. Also, in some embodiments, each of the dimensions of the demineralized cancellous bone matrix are individually between about 0.5 mm to about 20, about 2 mm to about 12 mm, or about 5 mm to about 10 mm. Furthermore, in some embodiments, the size of the individual dimensions can vary along the dimensions. In certain embodiments, about 100%, or at least 95%, 90% or 80% or the majority of the demineralized cancellous bone matrices of an implant have dimensions, including but not limited to a length, between about 0.5 to about 20 mm, between about 2 mm to about 12 mm, or between about 5 mm to about 10 mm.

Moreover, a demineralized cancellous bone matrix can have various shapes. For instance the demineralized cancellous bone matrix can have a cylindrical, spherical, pyramidal, ovoid, discoid, oblong or cuboidal shape. In other embodiments, at least one surface of the demineralized cancellous bone matrix has an irregular polygonal shape or regular polygonal shape. In some embodiments, the implant comprises more than one demineralized cancellous bone matrix, where the demineralized cancellous bone matrices have the same shape. In other embodiments, an implant comprises more than one demineralized cancellous bone matrix, where the demineralized cancellous bone matrices have different shapes.

In certain embodiments, the demineralized cancellous bone matrix is formed from human bone. In other embodiments, the demineralized cancellous bone matrix is formed from non-human bone. In certain embodiments, the demineralized cancellous bone matrix is obtained from autograft, allograft or xenogeneic sources. In some embodiments, the demineralized cancellous bone matrix is obtained from a cadaver.

Moreover, in certain embodiments, the demineralized cancellous bone matrix or implant further comprises at least one additional component, such as bone marrow aspirate, blood, platelet rich plasma, platelet rich plasma matrix, one or more cells (such as stem cells, epithelial cells, fibroblasts, osteoblasts, osteoclasts), chemotactic factors, growth factors (such as BMP, VEGF, IGF, PDGF and EGF), carriers (such as saline, phosphate buffered solution, sodium hyaluronate, hyaluronic acid), non-demineralized or demineralized cortical bone, or combinations thereof. In certain embodiments, the demineralized cancellous bone matrix comprises mesenchymal stem cells. Moreover, in certain embodiments, the BMP can comprise for example, BMP-2, BMP-4, or combinations thereof. Mesenchymal stem cells can be seeded onto the surface of the demineralized cancellous bone matrix. In some embodiments, the at least one additional component induces or accelerates new bone formation. In some embodiments, the at least one additional component is osteogenic, osteoinductive and/or osteoconductive.

In some embodiments, the demineralized cancellous bone matrix includes about 0% to about 50% by weight of cortical bone. In certain embodiments, the demineralized cancellous bone matrix includes less than or equal to about 10%, about 5%, about 1%, or about 0.5% by weight of cortical bone. In yet other embodiments, the demineralized cancellous bone matrix includes about 0% to about 95% by weight of growth factors. Also in some embodiments, the demineralized cancellous bone matrix includes less than or equal to about 10%, about 5%, about 1%, or about 0.5% by weight of growth factors. Furthermore, in certain embodiments, the demineralized cancellous bone matrix includes about 0% to about 50% by weight of connective tissue. In certain embodiments, the demineralized cancellous bone matrix includes less than or equal to about 10%, about 5%, about 1%, or about 0.5% by weight of connective tissue. In some embodiments, the demineralized cancellous bone matrix is substantially free of cortical bone, growth factors and/or connective tissue.

Also disclosed herein are methods of treating bone having a void or defect in a patient in need thereof comprising inserting into the void or defect at least one demineralized cancellous bone matrix as described herein. For instance, the at least one demineralized cancellous bone matrix may comprise cancellous bone matrix that has been demineralized to contain about 5% to about 20% by weight residual calcium, or about 6% to about 15% by weight residual calcium, or any other range of residual calcium, such as those described herein. In certain embodiments, the at least one demineralized cancellous bone matrix may be rigid and can have dimensions, including a length, of between about 0.5 mm to about 20 mm. Also, in some embodiments, the dimensions of the demineralized cancellous bone matrix are individually between about 0.5 mm to about 20 mm. In certain embodiments, the at least one demineralized cancellous bone matrix can have a cuboidal shape or any other shape such as those described herein. Additionally, in some embodiments, at least one surface of the demineralized cancellous bone matrix can have an irregular or regular polygonal shape. Also, the at least one demineralized cancellous bone matrix can be formed from human bone or from non-human bone.

The demineralized cancellous bone matrix used in the method of treating bone having a void or defect may further comprise at least one additional component, including those described herein. In certain embodiments, the at least one demineralized cancellous bone matrix used in the method of treating bone having a void or defect includes certain amounts of cortical bone, growth factors, and/or connective tissue, including those described herein. Furthermore, the demineralized cancellous bone matrix used to treat bone having a void or defect can adsorb and/or wick blood.

In certain embodiments, in the method of treating bone having a void or defect, the demineralized cancellous bone matrix promotes bone growth in the void or defect. The void or defect in the bone treated using the methods disclosed herein can be related to a trauma or a cancer, or any other medical defect or condition that can benefit from such treatment.

Moreover, described herein are methods of treating spinal discs in a patient in need thereof comprising forming at least one cavity located between two adjacent vertebral bodies and inserting into the cavity at least one demineralized cancellous bone matrix described herein. In certain embodiments, the cavity is located within the spinal disc. In some embodiments, the at least one demineralized cancellous bone matrix promotes bone growth in the cavity. Also, in certain embodiments, the at least one demineralized cancellous bone matrix is used to create a fusion between the two vertebral bodies (e.g. adjacent vertebral bodies). In some embodiments, the spinal disc is degenerated and the method is for treating the degenerated spinal disc.

Furthermore, disclosed herein are methods of making the demineralized cancellous bone matrices. In certain embodiments, the method comprises the steps of cutting bone to obtain the cancellous bone matrix. The bone can be human or non-human. Also, in certain embodiments, the cancellous bone matrix is rigid. In some embodiments, the cancellous bone matrix has a length or other dimensions between about 0.5 mm to about 20 mm. Also, in some embodiments, each of the dimensions of the demineralized cancellous bone matrix are individually between about 0.5 mm to about 20 mm. In addition, in some embodiments, the size of the individual dimensions can vary along the dimension. Moreover, cancellous bone matrix can be cut to any shape. After the cancellous bone matrix has been obtained, the cancellous bone matrix can be demineralized to a certain residual calcium level. For instance, the cancellous bone matrix can be demineralized so that the residual calcium in the cancellous bone matrix is about 5% to about 20% by weight, or another residual calcium level as described herein.

The demineralization of the cancellous bone matrix can be achieved using an acid, e.g., hydrochloric acid. For example, about 0.2N to about 1.0N or about 0.3N to about 0.6N hydrochloric acid. The cancellous bone matrix may be demineralized for a certain amount of time. For instance, it can be demineralized for about 2 minutes to about 15 minutes, or for about 5 minutes to about 10 minutes to obtain the demineralized cancellous matrix described herein. In one specific embodiment, the cancellous bone matrix is demineralized for about 5 minutes to about 10 minutes in about 0.2N to about 0.5N hydrochloric acid. In another specific embodiment, the cancellous bone matrix is demineralized for about 4 minutes to about 10 minutes in about 0.2N to about 0.7N HCl.

The method of making the demineralized cancellous bone matrix can further comprise exposing the cancellous bone matrix to one or more bone cleaning agents before and/or after demineralization. In one embodiment, the cancellous bone matrix is exposed to the one or more bone cleaning agents before demineralizing the cancellous bone matrix.

In another embodiment, the demineralized cancellous bone matrix is exposed to the one or more bone cleaning agents after demineralization. In a further embodiment, the demineralized cancellous bone matrix is exposed to one or more bone cleaning agents before and after demineralization. The one or more bone cleaning agents can comprise, for example, hydrogen peroxide, a detergent, an antibiotic, an alcohol, or combinations thereof. In certain embodiments, the demineralized cancellous bone matrix can be sonicated in one or more bone cleaning agents. In some embodiments, the demineralized cancellous bone matrix can be agitated in one or more bone cleaning agents. In one embodiment, the demineralized cancellous bone matrix can be agitated by mechanical stirring. In a further embodiment the demineralized cancellous bone matrix can be soaked in one or more bone cleaning agents.

The method of making the demineralized cancellous bone matrix can further comprise combining the demineralized cancellous bone matrix with at least one additional component. The at least one additional component can comprise, for example, bone marrow aspirate, blood, platelet rich plasma, platelet rich plasma matrix, one or more cells (such stem cells, epithelial cells, fibroblasts, osteoblasts and osteoclasts), chemotactic factors, growth factors (such as BMP, VEGF, IGF, PDGF and EGF), carriers (such as saline, phosphate buffered solution, sodium hyaluronate, hyaluronic acid), non-demineralized or demineralized cortical bone, or combinations thereof. In a specific embodiment, the method of making the demineralized cancellous bone matrix comprises combining the demineralized cancellous bone matrix with mesenchymal stem cells. In certain embodiments, the method comprises combining the demineralized cancellous bone matrix with a BMP, which comprises BMP-2, BMP-4, or a combination thereof.

The process of making the demineralized cancellous bone matrix can further include modifying the amounts of certain substances, for example, cortical bone, growth factors and/or connective tissue, present in the demineralized cancellous bone matrix. For example, cortical bone, growth factors and/or connective tissue can be removed from the demineralized cancellous bone matrix until the desired amount of these materials in the demineralized cancellous matrix is obtained. In some embodiments, the process of making the demineralized cancellous bone matrix can yield demineralized cancellous bone matrix that includes less than or equal to about 10%, about 5%, or about 1% by weight of cortical bone, growth factors and/or connective tissue. Also, in certain embodiments, the process of making the demineralized cancellous bone matrix can yield demineralized cancellous bone matrix that includes about 0% to about 50% by weight cortical bone, about 0% to about 95% by weight growth factors, and/or about 0% to about 50% by weight of connective tissue.

The process of making the demineralized cancellous bone matrix can further include a step of testing blood adsorption or blood wicking capability of the demineralized cancellous bone matrix. The process of making the demineralized cancellous bone matrix can yield bone matrix that is osteoinductive, osteoconductive and/or osteogenic, and that can induce or accelerate new bone growth.

### BRIEF DESCRIPTION OF THE DRAWINGS

FIGS. 1A and 1B present a perspective view of a plurality of demineralized cancellous bone matrices that are box, cube, cylinder, disc, sphere or pyramid shaped.

FIG. 1B shows the demineralized cancellous bone matrices contained in a fill tube container. The demineralized cancellous bone matrices are situated in a single row in the fill tube container so that the demineralized cancellous bone matrices can be dispensed from the fill tube container a single-file order.

FIG. 2A shows a femur having a bone void. The bone void is being filled with demineralized cancellous bone matrices. FIG. 2B shows a cavity within a vertebral body having a compression or burst fracture. An implantable container, which has been placed within the cavity, is being filled with demineralized cancellous bone matrices from a delivery container.

FIG. 3 shows a cavity located between two adjacent vertebral bodies. An implantable container, which has been placed within the cavity, is being filled with demineralized cancellous bone matrices from a delivery container.

FIG. 4 (upper panels) shows blood adsorption by non-demineralized, demineralized (having about 6% to about 15% by weight residual calcium) and fully demineralized cancellous bone matrices, after pipetting five drops of blood on top of the cancellous bone matrices. FIG. 4 (lower panels) shows blood wicking capacity of non-demineralized, demineralized (having about 6% to about 15% by weight residual calcium) and fully demineralized cancellous bone matrices, after placing the cancellous bone matrices on three drops of blood.

FIG. 5 is a scanning electron microscopy image showing adhesion of mesenchymal stem cells on the surface of non-demineralized, demineralized (having about 6% to about 15% by weight residual calcium) and fully demineralized cancellous bone matrices at 12 hours after seeding of the cells.

FIG. 6 is a multiphoton microscopy image showing cell proliferation of mesenchymal stem cells labeled with Alexa Fluor 633 and CMFDA (5-chlormethylfluorescein diacetate) on the surface of non-demineralized, demineralized (having about 6% to about 15% by weight residual calcium) and fully demineralized cancellous bone matrices at 5 days after seeding of the cells.

FIG. 7 shows a histology sample of mesenchymal stem cells labeled with H&E (hematoxylin and eosin) on the surface of non-demineralized, demineralized (having about 6% to about 15% by weight residual calcium) and fully demineralized cancellous bone matrices at 2 weeks after seeding of the cells.

FIG. 8 shows total DNA quantification in mesenchymal stem cells per unit of non-demineralized, fully demineralized and demineralized (having about 6% to about 15% by weight residual calcium) cancellous bone matrices at 3 days, 1 week and 2 weeks post-seeding.

FIG. 9 shows quantification of BMP-2 protein levels per gram of demineralized cancellous bone matrix (having about 6% to about 15% by weight residual calcium) in samples from four different donors.

FIG. 10 shows levels of alkaline phosphatase per 1 ng of total DNA in mesenchymal stem cells cultured on non-demineralized, fully demineralized or demineralized (having about 6% to about 15% by weight residual calcium) cancellous bone matrices at 1 week or 2 weeks post-seeding.

### DETAILED DESCRIPTION OF THE INVENTION

### Definitions

As used herein, and unless otherwise defined, the term “cancellous bone matrix” refers to a unit of bone that is made of all or substantially all cancellous bone.

As used herein, and unless otherwise defined, the term “demineralized,” when used in connection with bone, refers to bone that has had at least a portion of its calcium content removed.

As used herein, and unless otherwise defined, the term “fully demineralized,” when used in connection with bone, refers to bone that has had calcium removed from the bone so that the residual calcium content is less than or equal to about 0.5 weight percent of the bone.

As used herein, and unless otherwise defined, the term “non-demineralized,” when used in connection with bone, refers to bone that has not had calcium removed from the bone.

As used herein, and unless otherwise defined, the term “rigid,” when used in connection with the demineralized cancellous bone matrix, refers to demineralized cancellous bone matrix that has structural firmness and has substantially the same dimensions under compression.

As used herein, and unless otherwise defined, the term “length,” when used in connection with demineralized cancellous bone matrix, refers to the largest dimension of the demineralized cancellous bone matrix.

As used herein, and unless otherwise defined, the term “dimension” or “dimensions” when used in connection with demineralized cancellous bone matrix, refers to the length, height, width, diameter or any other measurement of the size of the demineralized cancellous bone matrix.

As used herein, and unless otherwise defined, the term “individual dimension” or “individual dimensions” when used in connection with demineralized cancellous bone matrix, refers to the length, height, width, diameter or any other measurement of the size of the demineralized cancellous bone matrix, wherein the size of the specific dimension can vary along the dimension.

As used herein, and unless otherwise defined, the term “cuboidal shape,” when used in connection with demineralized cancellous bone matrix, refers to a matrix having six sides, in which the six sides are in the shape of a quadrilateral or four-sided polygon.

As used herein, and unless otherwise defined, the term “regular polygonal shape,” when used in connection with a surface of the demineralized cancellous bone matrix, refers to the surface having a polygonal shape in which the sides of the polygonal shape have about the same length.

As used herein, and unless otherwise defined, the term “irregular polygonal shape,” when used in connection with a surface of the demineralized cancellous bone matrix, refers to the surface having a polygonal shape in which the sides of the polygonal shape can have different lengths.

As used herein, and unless otherwise defined, the term “non-human bone” refers to bone obtained from an organism that is not a human.

As used herein, and unless otherwise defined, the term “void or defect related to a trauma or a cancer,” when used in connection with a bone, refers to a void or defect in the bone that resulted from a trauma or a cancer, or from a treatment for a trauma or cancer.

As used herein, and unless otherwise defined, the term “bone cleaning agent” refers to a substance used to remove unwanted matter from bone.

### Cancellous Bone Matrix

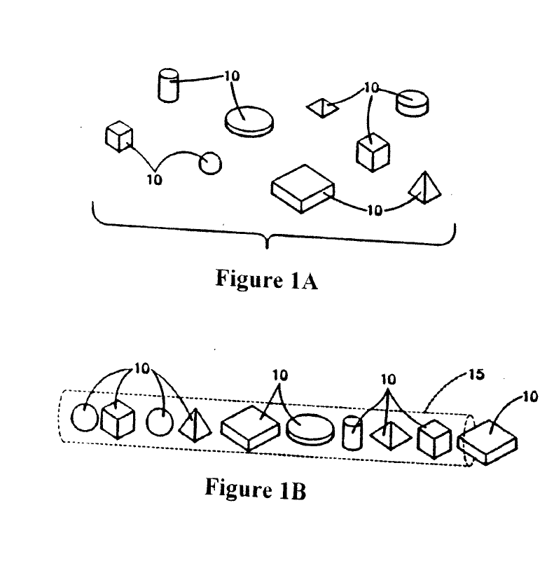

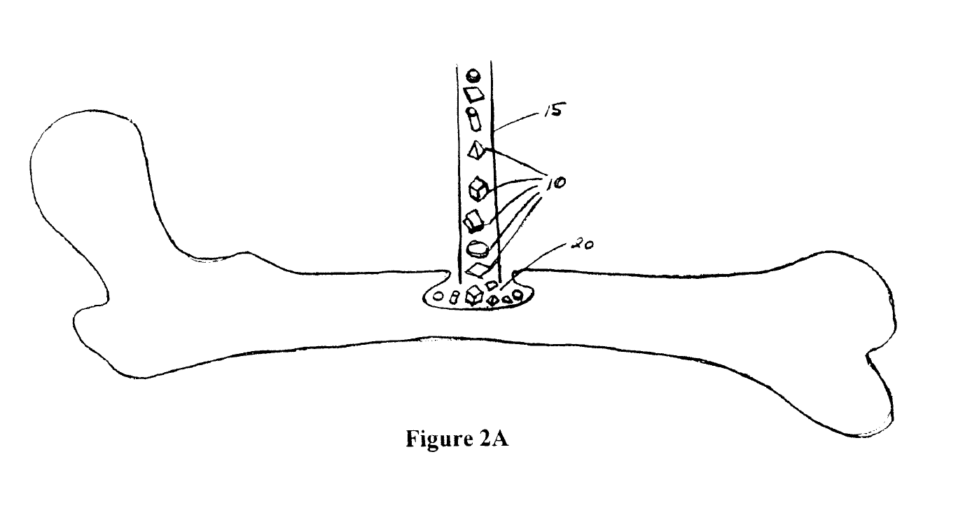

FIG. 1A shows a plurality of demineralized cancellous bone matrices 10. As shown in this figure, the demineralized cancellous bone matrices 10 can be discrete or not connected to each other. FIG. 1B shows an example of a plurality of the demineralized cancellous bone matrices 10 contained in a delivery container 15, such as a delivery tube or cannula, which can facilitate the delivery and implantation of the demineralized cancellous bone matrices 10 to a patient. In this embodiment, the demineralized cancellous bone matrices 10 are situated in a single row in the delivery container 15. In other embodiments, the demineralized cancellous bone matrices can be situated in the delivery container in different arrangements.

As shown in FIG. 1A, the demineralized cancellous bone matrices 10 can have a variety of geometric shapes. For example, the demineralized cancellous bone matrices may have a particular shape, including, but not limited to, a cylindrical, spherical, pyramidal, ovoid, discoid, oblong (i.e., box) or cuboidal shape. At least one surface of the demineralized cancellous bone matrix may have an irregular polygonal shape or a regular polygonal shape. In addition, in an implant comprising more than one demineralized cancellous bone matrix, the demineralized cancellous bone matrices can have the same shape or a variety of shapes. For instance, at least one of the demineralized cancellous bone matrices can have a particular shape, while other demineralized cancellous bone matrices can have the same or different shapes.

In certain embodiments, the demineralized cancellous bone matrix is rigid. In some embodiments, the volume of the demineralized cancellous bone matrix decreases under compression about 0% to about 5%. Furthermore, in certain embodiments, the volume of the demineralized cancellous bone matrix decreases under compression about 5% or less, about 4% or less, about 3% or less, about 2% or less, about 1% or less, or about 0.5% or less.

In some embodiments, the demineralized cancellous bone matrix has dimensions, including but not limited to a length, between about 0.1 mm to about 20 mm about 0.5 mm to about 20 mm, about 0.5 mm to about 15 mm, about 0.5 mm to about 12 mm, about 0.5 mm to about 10 mm, about 0.75 mm to about 20 mm, about 0.75 mm to about 14 mm. 0.75 mm to about 12 mm, about 0.75 mm to about 9 mm, about 0.85 mm to about 15 mm, about 0.85 mm to about 8 mm, about 1 mm to about 20 mm, about 1 mm to about 15 mm, about 1 mm to about 12 min, about 1 mm to about 10 mm, about 1 mm to about 9 mm, about 1 mm to about 8 mm, about 1 mm to about 7 mm, about 1 mm to about 6 mm, about 1.5 min to about 20 mm, about 1.5 mm to about 12 mm, about 1.5 mm to about 10 mm, about 1.5 mm to about 5 mm, about 1.5 mm to about 4 mm, about 1.5 mm to about 3 mm, about 2 mm to about 20 mm, about 2 mm to about 12 mm, about 2 mm to about 10 mm, about 2 mm to about 8 mm, about 2 mm to about 6 mm, about 2 mm to about 4 mm, about 3 mm to about 20 mm, about 3 mm to about 10 mm, about 3 mm to about 9 mm, about 3 mm to about 8 mm, about 4 mm to about 20 mm, about 4 mm to about 10 mm, about 4 mm to about 8 mm, about 5 mm to about 20 mm, about 5 mm to about 10 mm, or about 5 mm to about 7 mm.

In addition, in some embodiments, the demineralized cancellous bone matrix has dimensions, including but not limited to a length, greater than or equal to: about 0.1 mm, about 0.25 mm, about 0.5 mm, about 0.75 mm, about 0.8 mm, about 0.85 mm, about 0.9 mm, about 0.95 mm, about 1.0 mm, about 1.25 mm, about 1.5 mm, about 1.75 mm, about 2.0 mm, about 2.25 mm, about 2.5 mm, about 2.75 mm, about 3.0 mm, about 3.25, about 3.5 mm, about 3.75 mm, about 4.0 mm, about 4.25 mm, about 4.5 mm, about 4.75 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm or about 10.0 mm.

In certain embodiments, the demineralized cancellous bone matrix has dimensions, including but not limited to a length, less than or equal to: about 0.1 mm, about 0.25 mm, about 0.5 mm, about 0.75 mm, about 0.8 mm, about 0.85 mm, about 0.9 mm, about 0.95 mm, about 1.0 mm, about 1.25 mm, about 1.5 mm, about 1.75 mm, about 2.0 mm, about 2.25 mm, about 2.5 mm, about 2.75 mm, about 3.0 mm, about 3.25, about 3.5 mm, about 3.75 mm, about 4.0 mm, about 4.25 mm, about 4.5 mm, about 4.75 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.0 mm, about 15.0 or about 20.0 mm.

Furthermore, in some embodiments, when more than one demineralized cancellous bone matrix is involved, a certain weight percent of the demineralized cancellous bone matrices have certain dimensions, including but not limited to length, that can be between about 0.5 mm to about 20 mm. For example, the demineralized cancellous bone matrices may contain about 0.5% to about 95% by weight, about 5% to about 95% by weight, about 10% to about 85% by weight, about 15% to about 75% by weight, or about 25% to about 50% by weight of demineralized cancellous bone matrices having dimensions, including but not limited to length, between about 0.5 mm to about 20 mm. In certain embodiments, the demineralized cancellous bone matrices may contain greater than or equal to: about 0.5% by weight, about 1% by weight, about 5% by weight, about 10% by weight, about 15% by weight, about 20% by weight, about 25% by weight, about 30% by weight, about 40% by weight, about 50% by weight, about 60% by weight, about 70% by weight, about 80% by weight, about 90% or about 95% by weight of demineralized cancellous bone matrices having dimensions, including but not limited to a length, between about 0.5 mm to about 20 mm.

The bone used to form the demineralized cancellous bone matrices can be demineralized. The bone can be cleaned before and/or after it is demineralized. Also, the bone can be demineralized before or after it is milled into the cancellous bone matrices. For example, the bone may be cut or milled into the cancellous bone matrices having the desired shape prior to the demineralization process. In other embodiments, the bone can be demineralized before being cut or milled into the cancellous bone matrices having the desired shape.

To demineralize the bone, the bone is placed in acid. In certain embodiments, the bone can be demineralized such that it contains residual calcium in an amount of about 1% by weight to about 10% by weight, about 1% by weight to about 15% by weight, about 1% by weight to about 20% by weight, about 1% by weight to about 25% by weight, about 1% by weight to about 35% by weight, about 1% by weight to about 50% by weight, about 1% by weight to about 75% by weight, about 1% by weight to about 95% by weight, about 5% by weight to about 10% by weight, about 5% by weight to about 15% by weight, about 5% by weight to about 20% by weight, about 5% by weight to about 25% by weight, about 5% by weight to about 35% by weight, about 5% by weight to about 50% by weight, about 5% by weight to about 75% by weight, about 5% by weight to about 95% by weight, about 6% by weight to about 10% by weight, about 6% by weight to about 15% by weight, about 6% by weight to about 20% by weight, about 6% by weight to about 25% by weight, about 6% by weight to about 35% by weight, about 6% by weight to about 50% by weight, about 6% by weight to about 75% by weight, about 6% by weight to about 95% by weight, about 7% by weight to about 10% by weight, 7% by weight to about 12% by weight, about 7% by weight to about 15% by weight, about 7% by weight to about 20% by weight, about 7% by weight to about 25% by weight, about 7% by weight to about 35% by weight, about 7% by weight to about 50% by weight, about 7% by weight to about 75% by weight, about 7% by weight to about 95% by weight, about 8% by weight to about 10% by weight, about 8% by weight to about 12% by weight about 8% by weight to about 15% by weight, about 8% by weight to about 20% by weight, about 8% by weight to about 25% by weight, about 8% by weight to about 35% by weight, about 8% by weight to about 50% by weight, about 8% by weight to about 75% by weight, about 8% by weight to about 95% by weight; about 10% by weight to about 95% by weight; about 20% by weight to about 95% by weight; about 50% by weight to about 95% by weight or about 50% by weight to about 75% by weight. In a particular embodiment, the bone can be demineralized such that it contains residual calcium in an amount of about 6% by weight to about 15% by weight.

In certain embodiments, the bone can be demineralized such that it contains residual calcium in an amount greater than or equal to: about 1% by weight, about 2% by weight, about 3% by weight, about 4% by weight, about 5% by weight, about 6% by weight, about 7% by weight, about 8% by weight, about 9% by weight, about 10% by weight, about 15% by weight, about 20% by weight, about 25% by weight, about 30% by weight, about 35% by weight, about 40% by weight, about 45% by weight, about 50% by weight, about 55% by weight, about 60% weight, about 65% by weight, about 70% weight, about 75% by weight, about 80% by weight, about 85% by weight, about 90% by weight, or about 95% by weight.

The demineralized cancellous bone matrices can contain the amount of residual calcium that renders such matrices rigid. In one embodiment, the demineralized cancellous bone matrices remain rigid in the presence of a liquid, such as saline or body fluids.

Also, the demineralized cancellous bone matrices may be derived from autograft bone, allograft bone, or xenograft bone. In particular embodiments, the demineralized cancellous bone matrices are derived from allograft bone. In some embodiments, the demineralized cancellous bone matrices are derived from a mammal, such as a human. In certain embodiments, the demineralized cancellous bone matrices are derived from non-human bone. The cancellous bone used to make the demineralized cancellous bone matrices may be derived from any bone, including, but not limited to, the cranium, femur, tibia, humerus, fibula, radius, and ulna. In a specific embodiment, the demineralized cancellous bone matrices are derived from a cadaver, such as a human cadaver or a non-human cadaver.

Moreover, in some embodiments, the demineralized cancellous bone matrices are made exclusively or primarily of cancellous bone. In some embodiments, the demineralized cancellous bone matrices can include cancellous bone in an amount about 5% to about 100%, about 25% to about 100% by weight, about 50% to about 100% by weight, about 75% to about 100% by weight, about 85% to about 100% by weight, about 90% to about 100% by weight, or about 95% to about 100% by weight. In certain embodiments, the demineralized cancellous bone matrices can comprise cancellous bone in an amount greater than or equal to: about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight.

Also, the demineralized cancellous bone matrices described herein may be free of cortical bone or substantially free of cortical bone. In certain embodiments, the demineralized cancellous bone matrices can comprise cortical bone in an amount of about 0% to about 95%, about 0% to about 50% by weight, about 0% to about 25% by weight, about 0% to about 10% by weight, about 0% to about 5% by weight, about 0% to about 2.5% by weight, or about 0% to about 1% by weight. Also, in some embodiments, the demineralized cancellous bone matrices can comprise cortical bone in an amount less than or equal to: about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. Also, the cortical bone can have dimensions, including but not limited to length, that can be between about 0.5 mm to about 20 mm.

The demineralized cancellous bone matrices described herein are preferably osteoinductive. The demineralized cancellous bone matrices may also be osteoconductive. The osteoinductive and/or osteoconductive nature of the demineralized cancellous bone matrices described herein may engender biological repair of a bone void, defect or damaged vertebral body with new bone formation and tissue remodeling.

The cancellous bone used to prepare the demineralized cancellous bone matrices can be cleaned to eliminate undesired substances. These undesired substances can include without limitation lipids, cells and microorganisms, e.g., viruses, bacteria. The bone can be cleaned by exposing it to a detergent or an agent that eliminates microorganisms, such as an antibiotic, an alcohol, e.g., ethanol, or hydrogen peroxide. The demineralized cancellous bone matrices can be exposed to one or more bone cleaning agents, such as hydrogen peroxide, a detergent, an antibiotic or an alcohol.

Also, the demineralized cancellous bone matrices described herein may be free or substantially free of non-demineralized, i.e., mineralized bone. In some embodiments, the demineralized cancellous bone matrices can comprise non-demineralized bone in an amount of about 0% to about 90% by weight, about 0% to about 75% by weight, about 0% to about 50% by weight, about 0% to about 25% by weight, about 0% to about 10% by weight, about 0% to about 5% by weight, about 0% to about 1% by weight, about 4% to about 25% by weight, about 4% to about 20% by weight, about 4% to about 15% by weight, about 4% to about 14% by weight, about 4% to about 10%, about 5% to about 25% by weight, about 5% to about 20% by weight, about 5% to about 18% by weight, about 5% to about 15% by weight, about 5% to about 12% by weight, about 5% to about 10% by weight, about 6% to about 25% by weight, about 6% to about 20% by weight, about 6% to about 15% by weight, about 6% to about 12% by weight, about 6% to about 10% by weight, about 7% to about 20% by weight, about 7% to about 15% by weight, or about 7% to about 12% by weight. Furthermore, in certain embodiments the demineralized cancellous bone matrices can comprise non-demineralized bone in an amount less than or equal to: about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight.

In some embodiments, the demineralized cancellous bone matrices can be radiopaque. In particular embodiments, the demineralized cancellous bone matrices will be radiopaque before or during implantation into a subject. In such embodiments, the demineralized cancellous bone matrices will be seen in x-rays immediately upon implantation.

In certain embodiments, the demineralized cancellous bone matrices can be radiolucent. In particular embodiments, the demineralized cancellous bone matrices will be radiolucent before or during implantation into a subject. In such embodiments, the demineralized cancellous bone matrices will not be seen in x-rays immediately upon implantation. After time, as new bone grows, the implant site will become radiopaque and will be visible in X-rays as a way of tracking the patient's bone growth. In some embodiments, as the demineralized cancellous bone matrices begin to remodel and new bone begins to form, the demineralized cancellous bone matrices may be radiolucent from the time of implantation for about 2 weeks to about 6 months, for about 2 weeks to about 32 weeks, for about 4 weeks to about 28 weeks, for about 6 weeks to about 24 weeks, or for about 6 weeks to about 12 weeks. Also, in certain embodiments the demineralized cancellous bone matrices may be radiolucent from the time of implantation: up to about 2 weeks, up to about 4 weeks, up to about 6 weeks, up to about 8 weeks, up to about 10 weeks, up to about 12 weeks, up to about 15 weeks, up to about 18 weeks, up to about 24 weeks, up to about 28 weeks, or up to about 32 weeks.

In yet other embodiments, the demineralized cancellous bone matrices described herein may include the addition of a radiopaque marker to the demineralized cancellous bone matrices in order to make the demineralized cancellous bone matrices visible during surgery. The radiopaque marker may be derived from, but is not limited to, beryllium copper, brass, bronze, carbon steel, clad metals, copper, kovar, molybdenum, nickel, niobium, stainless steel, tantalum, titanium, zirconium, or other radiopaque material. Other suitable materials may include, without limitation, barium, platinum, platinum iridium, gold, and iodine-containing compounds. In a particular embodiment, the radiopaque marker may be incorporated into the demineralized cancellous bone matrices as a separate unit in the form of a pellet or wire. In another embodiment, radiopacity may be attained by chemically binding a radiopaque marker to single or multiple demineralized cancellous bone matrices prior to implantation. The radiopaque marker may be permanent or have a temporary lifetime. In certain embodiments in which the radiopaque marker has a temporary lifetime, it has a temporary lifetime of about one month to about one year. In some embodiments in which the radiopaque marker has a temporary lifetime, it has a temporary lifetime of at least one month, at least two months, at least three months, at least four months, at least five months, at least six months, or at least one year.

As seen in FIG. 1B, a plurality of demineralized cancellous bone matrices 10 may be contained in a delivery container 15, such that the demineralized cancellous bone matrices are capable of being dispensed from the delivery container in a single-file order. It can be advantageous for the demineralized cancellous bone matrices to be of a size and shape that enables them to be dispensed in a single-file order. This avoids problems of the demineralized cancellous bone matrices sliding, wedging, and jamming during delivery of the demineralized cancellous bone matrices to the implantation site from the delivery container. The delivery container may be, without limitation, a fill tube, a syringe, a cannula, a cartridge, a hollow rod, or a hollow delivery tube. In FIG. 1B, the delivery container 15 is depicted as a fill tube. The delivery container may vary in diameter. In some embodiments, the delivery container has a diameter about 0.5 mm to about 30 mm, of about 1 mm to about 15 mm, about 1 mm to about 10 mm, or about 2 mm to about 8 mm. Furthermore, the container can be made of a radiopaque material or have at least one radiopaque marker, which would make the container visible during implantation, even though the demineralized cancellous bone matrices are radiolucent.

In other embodiments, the demineralized cancellous bone matrices may include one or more additional components. For example, the additional component can be the radiopaque marker described herein. The additional component can be non-demineralized or demineralized cortical bone. In addition, the additional component can be a carrier.

In some embodiments, the demineralized cancellous bone matrices described herein are free of one or more additional components. Alternatively, the demineralized cancellous bone matrices can include one or more additional components, including those described herein, in an amount of about 0.1 to about 95% by weight, about 1% to about 25% by weight, about 1% to about 10% by weight, about 5% to about 25% by weight, about 5% to about 10% by weight, about 10% to about 90% by weight, about 20% to about 85% by weight, about 30% to about 80% by weight, or about 50% to about 75% by weight. In certain embodiments, the demineralized cancellous bone matrices can include one or more additional components, including those described herein, in an amount less than or equal to about 0.1% by weight, 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight.

In embodiments having a carrier, the demineralized cancellous bone matrices can be mixed with the carrier. Additionally, the carrier may act to preserve osteoinductivity of the demineralized cancellous bone matrices and/or provide other biological effects, e.g., support vascularization. The carrier can be biocompatible and/or biodegradable. Also, the carrier can be used to rehydrate the demineralized cancellous bone matrices. Therefore, in certain embodiments, the carrier may comprise a hydrating agent. In some embodiments, the demineralized cancellous bone matrices may be suspended in the carrier. In other embodiments, the carrier may be adsorbed by the demineralized cancellous bone matrices so that surfaces of the demineralized cancellous bone matrices are surrounded by no carrier or only small amounts of a carrier.

In some embodiments, the carrier can comprise a lubricant to reduce or eliminate any friction between the demineralized cancellous bone matrices and the devices used to deliver the demineralized cancellous bone matrices to an implantation site. For instance, the carrier comprising a lubricant may facilitate loading of the demineralized cancellous bone matrix into a delivery container, such as a fill tube, as well as delivery of the demineralized cancellous bone matrices from the delivery container during implantation. Also, the carrier comprising a lubricant can reduce or eliminate the friction among the demineralized cancellous bone matrices.

The carrier may be, without limitation, saline, phosphate buffered solution, phosphate buffered saline, sodium hyaluronate, hyaluronic acid, alginate, dextran, gelatin, collagen, glycerin, glycine, glycerol, polyethylene glycol, oils, fatty acids, saccharides, polysaccharides, glycoproteins, water soluble polymers, or combinations thereof. In particular embodiments, the carrier is sodium hyaluronate.

In certain embodiments, the demineralized cancellous bone matrix or matrices can include a carrier in an amount of about 0.1% to about 95%, about 1% to about 25% by weight, about 1% to about 10% by weight, about 5% to about 25% by weight, about 5% to about 10% by weight, about 10% to about 90% by weight, about 20% to about 85% by weight, about 30% to about 80% by weight, or about 50% to about 75%. Furthermore, the demineralized cancellous bone matrix or matrices can include a carrier in an amount less than or equal to about 0.1% by weight, 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight. In some embodiments, the demineralized cancellous bone matrix is free of a carrier.

In certain embodiments, the additional component can be a synthetic material, such as a material of similar physical dimensions as the demineralized cancellous bone matrices. Such synthetic material(s) include, but are not limited to, polymeric hydrogels, biodegradable polymers, rubbers, or other materials that are elastic in nature.

The additional component can also be cells and/or bioactive agents. The cells and/or bioactive agents can be added to the demineralized cancellous bone matrices, either prior to implantation or post-implantation. Supplementation with cells and/or bioactive agents may induce or accelerate new bone formation within a bone defect following implantation. Such cells may be transplanted cells, and may include, without limitation, autologous cells, allogenic cells, cells derived from bone marrow (e.g., bone marrow aspirate), stem cells (e.g., mesenchymal stem cells), other pluripotent cells, osteoblasts, osteoclasts, progenitor cells, chondrocytes, nucleus pulposus cells, epithelial cells, and fibroblasts. In a specific embodiment, the additional component is mesenchymal stem cells. Biological or bioactive agents may include, without limitation, viral particles, plasmids, proteins, hormones, extracellular matrix proteins, blood, platelet rich plasma, platelet rich plasma matrix, chemotactic factors, or growth factors. Examples of growth factors include without limitation those in the transforming growth factor (“TGF”), e.g., TGF-B; fibroblast growth factor (“FGF”); vascular endothelial growth factor (“VEGF”); insulin-like growth factor (“IGF”); platelet-derived growth factor (“PDGF”); and epithelial growth factor (“EGF”) and bone morphogenetic proteins (“BMP”).

The demineralized cancellous bone matrices may include one additional component, two additional components, three additional components, or more than three additional components.

The demineralized cancellous bone matrices may be free of one or more growth factors, cytokines and/or BMPs or substantially free of one or more growth factors, cytokines and/or BMPs. In some embodiments, the demineralized cancellous bone matrices may contain one or more BMPs (e.g., BMP-1, BMP-2, BMP-3, BMP-4, BMP-5, BMP-6, BMP-7 or BMP-8a). In some embodiment, the demineralized cancellous bone matrices may contain one or more growth factors and/or cytokines such as, but not limited to, TGF, FGF, VEGF, IGF, PDGF and EGF. In other embodiments, the demineralized cancellous bone matrices may contain one or more BMPs, but be free or substantially free of other growth factors and/or cytokines. In some embodiments, the demineralized cancellous bone matrices may be free or substantially free of exogenously added growth factors, cytokines and/or BMPs. Such demineralized cancellous bone matrices may contain endogenous growth factors, cytokines and/or BMPs present in the demineralized cancellous bone matrices.

In certain embodiments, the demineralized cancellous bone matrices can comprise growth factors, cytokines and/or BMPS in an amount of about 0 to about 95% by weight, about 0% to about 50% by weight, about 0% to about 25% by weight, about 0% to about 10% by weight, about 0% to about 5% by weight, about 0% to about 2.5% by weight, about 0% to about 1% by weight, about 0% to about 0.1% by weight, or about 0% to about 0.01% by weight. The demineralized cancellous bone matrices can comprise growth factors, cytokines and/or BMPs in an amount less than or equal to: about 0.01% by weight, about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight.

In some embodiments, the demineralized cancellous bone matrices can comprise exogenously added growth factors, cytokines and/or BMPs in an amount of about 0 to about 95% by weight, about 0% to about 50% by weight, about 0% to about 25% by weight, about 0% to about 10% by weight, about 0% to about 5% by weight, about 0% to about 2.5% by weight, about 0% to about 1% by weight, about 0% to about 0.1% by weight, or about 0% to about 0.01% by weight. In certain embodiments, the demineralized cancellous bone matrices can comprise exogenously added growth factors, cytokines, and/or BMPs in an amount less than or equal to: about 0.01% by weight, about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, to about 55% by weight, about 60% by weight, about 65% by weight, about 70% by weight, about 75% by weight, about 80% by weight, about 85% by weight, about 90% by weight, or about 95% by weight.

In embodiments where the demineralized cancellous bone matrices include one or more BMPs (e.g., BMP-1, BMP-2, BMP-3, BMP-4, BMP-5, BMP-6, BMP-7 or BMP-8a), the demineralized cancellous bone matrices may include about 0.01% to about 99%, about 0.01% to about 75%, about 0.01% to about 50%, about 0.01% to about 25% or about 0.01% to about 5% of the BMP proteins contained in the demineralized cancellous bone matrices before demineralization or estimated to be contained in the demineralized cancellous bone matrices before demineralization. In certain embodiments where the demineralized cancellous bone matrices include one or more BMPs (e.g., BMP-1, BMP-2, BMP-3, BMP-4, BMP-5, BMP-6, BMP-7 or BMP-8a), the demineralized cancellous bone matrices may include greater than or equal to about 0.01% about 0.1%, about 1%, about 5%, about 10%, about 15%, about 20%, about 25%, about 30%, about 50%, about 60%, about 75%, about 80%, about 85%, about 90%, about 95%, or about 99% of the BMPs contained in the demineralized cancellous bone matrices before demineralization or estimated to be contained in the demineralized cancellous bone matrices before demineralization.

Furthermore, in some embodiments, the demineralized cancellous bone matrices may contain about 0.01% to about 99%, about 0.01% to about 75%, about 0.01% to about 50%, about 0.01% to about 25% or about 0.01% to about 5% of endogenous (i.e., not exogenously added) BMPs contained in the demineralized cancellous bone matrices before demineralization or estimated to be contained in the demineralized cancellous bone matrices before demineralization. Also, in certain embodiments, the demineralized cancellous bone matrices may contain greater than or equal to: about 0.01% about 0.1%, about 1%, about 5%, about 10%, about 15%, about 20%, about 25%, about 30%, about 50%, about 60%, about 75%, about 80%, about 85%, about 90%, about 95%, or about 99% of endogenous BMP proteins contained in the demineralized cancellous bone matrices before demineralization or estimated to be contained in the demineralized cancellous bone matrices before demineralization.

In embodiments where the demineralized cancellous bone matrices include one or more growth factors and/or cytokines such as, but not limited to, VEGF, IGF, PDGF and EGF, the demineralized cancellous bone matrices may include about 0.01% to about 99%, about 0.01% to about 75%, about 0.01% to about 50%, about 0.01% to about 25% or about 0.01% to about 5% of the growth factors and/or cytokines contained in the demineralized cancellous bone matrices before demineralization or estimated to be contained in the demineralized cancellous bone matrices before demineralization. Also, in embodiments where the demineralized cancellous bone matrices include one or more growth factors and/or cytokines such as, but not limited to, VEGF, IGF, PDGF and EGF, the demineralized cancellous bone matrices may include greater than or equal to: about 0.01%, about 0.1%, about 1%, about 5%, about 10%, about 15%, about 20%, about 25%, about 30%, about 50%, about 60%, about 75%, about 80%, about 85%, about 90%, about 95%, or about 99% of the growth factors and/or cytokines contained in the demineralized cancellous bone matrices before demineralization or estimated to be contained in the demineralized cancellous bone matrices before demineralization.

In some embodiments, the demineralized cancellous bone matrices may contain about 0.01% to about 99%, about 0.01% to about 75%, about 0.01% to about 50%, about 0.01% to about 25% or about 0.01% to about 5% of endogenous (i.e., not exogenously added) growth factors and/or cytokines contained in the demineralized cancellous bone matrices before demineralization or estimated to be contained in the demineralized cancellous bone matrices before demineralization. Also, in certain embodiments, the demineralized cancellous bone matrices may contain greater than or equal to: about 0.01%, about 0.1%, about 1%, about 5%, about 10%, about 15%, about 20%, about 25%, about 30%, about 50%, about 60%, about 75%, about 80%, about 85%, about 90%, about 95%, or about 99% of endogenous (i.e., not exogenously added) growth factors and/or cytokines contained in the demineralized cancellous bone matrices before demineralization or estimated to be contained in the demineralized cancellous bone matrices before demineralization.

The demineralized cancellous bone matrices, in certain embodiments, may be free of connective tissue or substantially free of connective tissue. Moreover, in some embodiments, the demineralized cancellous bone matrices can comprise connective tissue in an amount about 0% to about 95% by weight, about 0% to about 50% by weight, about 0% to about 25% by weight, about 0% to about 10% by weight, about 0% to about 5% by weight, about 0% to about 2.5% by weight, or about 0% to about 1% by weight. In addition, in certain embodiments, the demineralized cancellous bone matrices can comprise connective tissue in an amount less than or equal to: about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. In some embodiments, the demineralized cancellous bone matrices can comprise exogenously added connective tissue in an amount about 0% to about 95% by weight, about 0% to about 50% by weight, about 0% to about 25% by weight, about 0% to about 10% by weight, about 0% to about 5% by weight, about 0% to about 2.5% by weight, or about 0% to about 1% by weight. In certain embodiments, the demineralized cancellous bone matrices can comprise exogenously added connective tissue in an amount less than or equal to: about 0.1% by weight, about 0.25% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, about 95% by weight, or about 100% by weight. The types of connective tissue contemplated herein include, without limitation, one or more of areolar or loose, adipose, dense, regular or irregular, white fibruous, elastic and cartilage connective tissue. The specific examples of connective tissue contemplated herein include, without limitation, one or more of fascia, skin, tendons, ligaments, pericardium and articular cartilage. In some embodiments, the demineralized cancellous bone matrices are free or substantially free of exogenously added calcium phosphate and/or collagen.

The demineralized cancellous bone matrices may be capable of adsorbing a biological fluid in an amount greater than the amount of the biological fluid adsorbed by non-demineralized or fully demineralized cancellous bone matrices. The biological fluid can include one type or more than one type of biological fluids. Biological fluids include, but are not limited to, blood, bone marrow, platelet rich plasma, platelet rich plasma matrix and adipose tissue aspirate.

The demineralized cancellous bone matrices may be capable of adsorbing a biological fluid (e.g., blood) quickly when exposed to drops of a biological fluid or a saturating amount of a biological fluid, for example, by placing an amount of blood on the top surface of the demineralized cancellous bone matrix. The demineralized cancellous bone matrices may be capable of adsorbing a biological fluid (e.g., blood) in about 0.1 second to about 1 minute, about 0.1 second to about 30 seconds, or about 0.1 second to about 15 seconds. Also, the demineralized cancellous bone matrices may be capable of adsorbing a biological fluid (e.g., blood) in equal to or less than about 0.1 second, about 0.25 seconds, about 0.5 seconds, about 0.75 seconds, about 1 second, about 1.5 seconds, about 2 seconds, about 2.5 seconds, about 3 seconds, about 4 seconds, about 5 seconds, about 6 seconds, about 7 seconds, about 8 seconds, about 10 seconds, about 12 seconds, about 15 seconds, about 20 seconds, about 30 seconds, or about 1 minute. The demineralized cancellous bone matrices described herein may be capable of adsorbing a biological fluid (e.g., blood) faster than non-demineralized and/or fully demineralized cancellous bone matrices. Specifically, the demineralized cancellous bone matrices described herein may be capable of adsorbing a biological fluid (e.g., blood) faster than non-demineralized and/or fully demineralized cancellous bone matrices of the same or substantially the same dimensions.

The demineralized cancellous bone matrices described herein may also be capable of superior wicking capacity for a biological fluid compared to non-demineralized and/or fully demineralized cancellous bone matrices. The biological fluid can include one type or more than one type of biological fluids. Biological fluids include, but are not limited to, blood, bone marrow, platelet rich plasma, platelet rich plasma matrix and adipose tissue aspirate. The demineralized cancellous bone matrix may display wicking capacity superior to that of non-demineralized and/or fully demineralized cancellous bone matrices of similar or same densities. The demineralized cancellous bone matrices may be capable of superior wicking capacity as may be manifested in the ability to draw a biological fluid (e.g., blood) vertically against the force of gravity by capillary action. The wicking capacity may be assessed by placing the demineralized cancellous bone matrices on top of a drop or several drops of blood, where the amount of blood can vary (e.g., a saturating amount of blood or non-saturating amount of blood).

The demineralized cancellous bone matrices may be capable of wicking or vertically drawing a biological fluid (e.g., blood), onto which the matrices are placed, further up the vertical dimension or height of the bone matrix than non-demineralized and/or fully demineralized cancellous bone matrices. Specifically, the demineralized cancellous bone matrices described herein may be capable of wicking or vertically drawing a biological fluid (e.g., blood) further up the vertical dimension or height of the bone matrix than non-demineralized and/or fully demineralized cancellous bone matrices of the same or substantially the same dimensions. In certain embodiments, the demineralized cancellous bone matrices may be capable of wicking a biological fluid about 1% to about 300%, about 1% to about 200%, about 1% to about 150%, about 1% to about 100%, or about 1% to about 50% further up the vertical dimension or height of the bone matrix than non-demineralized and/or fully demineralized cancellous bone matrices of the same or substantially the same dimensions. In some embodiments, the demineralized cancellous bone matrices may be capable of wicking a biological fluid greater than or equal to: about 1%, about 10%, about 20%, about 30%, about 50%, about 60%, about 70%, about 80%, about 90%, about 100%, about 110%, about 120%, about 130%, about 140%, about 150%, about 160%, about 170%, about 180%, about 190%, about 200%, about 250%, about or about 300% further up the vertical dimension or height of the bone matrix than non-demineralized and/or fully demineralized cancellous bone matrices of the same or substantially the same dimensions. Furthermore, in certain embodiments, the demineralized cancellous bone matrices may be capable of wicking a biological fluid (e.g., blood) to penetrate about 1% to about 100%, about 1% to about 75%, or about 1 to about 50% of the height and/or volume of the demineralized cancellous bone matrix. In some embodiments, the demineralized cancellous bone matrices may be capable of wicking a biological fluid (e.g., blood) to penetrate greater than or equal to: about 1%, about 10%, about 20%, about 30%, about 40%, about 50%, about 55%, about 60%, about 65%, about 70%, about 75%, about 80%, about 85%, about 90%, about 95%, about 99%, or about 100% of the height and/or volume of the demineralized cancellous bone matrix.

In certain embodiments, the demineralized cancellous bone matrices may also be capable of wicking a biological fluid (e.g., blood), onto which the matrix is placed, in about 0.1 second to about 10 minutes. Also, in some embodiments, the demineralized cancellous bone matrices may also be capable of wicking a biological fluid (e.g., blood), onto which the matrix is placed, in equal to or less than about 0.1 second, about 0.25 seconds, about 0.5 seconds, about 0.75 seconds, about 1 second, about 1.5 seconds, about 2 seconds, about 2.5 seconds, about 3 seconds, about 4 seconds, about 5 seconds, about 6 seconds, about 7 seconds, about 8 seconds, about 10 seconds, about 12 seconds, about 15 seconds, about 20 seconds, about 30 seconds, about 1 minute, about 2 minutes, about 3 minutes, about 4 minutes, about 5 minutes, about 6 minutes, about 7 minutes, about 8 minutes, about 9 minutes, or about 10 minutes. The demineralized cancellous bone matrices described herein may be capable of wicking a biological fluid (e.g., blood), onto which the matrix is placed, faster than non-demineralized and/or fully demineralized cancellous bone matrices. Specifically, the demineralized cancellous bone matrices described herein may be capable of wicking a biological fluid (e.g., blood) faster than non-demineralized and/or fully demineralized cancellous bone matrices of the same or substantially the same dimensions.

The demineralized cancellous bone matrices may promote cell adhesion, for example adhesion of stem cells, osteoclasts, osteoblasts, epithelial cells or fibroblasts to the surface of the demineralized cancellous bone matrices. The demineralized cancellous bone matrices may promote more cell adhesion than non-demineralized or fully demineralized cancellous bone matrices (e.g., when the cell adhesion capacity of bone matrices of the same or similar dimensions is compared). In some embodiments, the demineralized cancellous bone matrices may promote cell adhesion about 1% to about 99%, about 1% to about 80%, or about 1% to about 70% of the original number of cells, e.g., stem cells, seeded on the demineralized cancellous bone matrices. In certain embodiments, the demineralized cancellous bone matrices may promote cell adhesion of greater than or equal to about 1%, about 5%, about 10%, about 20%, about 30%, about 40%, about 50%, about 60%, about 70%, about 80%, about 90%, about 95%, or about 99% of the original number of cells, e.g., stem cells, seeded on the demineralized cancellous bone matrices. The demineralized cancellous bone matrices described herein may promote about 1% to about 300%, about 1% to about 200%, about 1% to about 150%, about 1% to about 100%, about 1% to about 75%, about 1% to about 50% or about 1% to about 25% more cell adhesion than non-demineralized or fully demineralized cancellous bone matrices, when the cells, e.g., stem cells, are seeded on the bone matrices. Also, the demineralized cancellous bone matrices described herein may promote about 1%, about 10%, about 20%, about 30%, about 40%, about 50%, about 60%, about 70%, about 75%, about 80%, about 90%, about 100%, about 150%, about 200%, about 300%, more cell adhesion than non-demineralized or fully demineralized cancellous bone matrices, when the cells, e.g., stem cells, are seeded on the bone matrices. In one embodiment, the demineralized cancellous bone matrices promote adhesion of mesenchymal stem cells.

The demineralized cancellous bone matrices may promote cell proliferation, for example proliferation of stem cells, osteoclasts, osteoblasts, epithelial cells or fibroblasts on the surface of the demineralized cancellous bone matrices. The demineralized cancellous bone matrices may promote more cell proliferation as compared to non-demineralized or fully demineralized cancellous bone matrices (e.g., when the cell proliferation on bone matrices of the same or similar dimensions is measured). In some embodiments, the demineralized cancellous bone matrices described herein may promote about 1% to about 300%, about 1% to about 200%, about 1% to about 150%, about 1% to about 100%, about 1% to about 75%, or about 1% to about 50%, or about 1% to about 25% more cell proliferation or faster rate of cell proliferation than non-demineralized or fully demineralized cancellous bone matrices, when the cells, e.g., stem cells, are seeded on the bone matrices. The demineralized cancellous bone matrices described herein may promote about 1%, about 5%, about 10%, about 20%, about 30%, about 40%, about 50%, about 60%, about 70%, about 75%, about 80%, about 90%, about 100%, about 150%, about 200%, or about 300% more cell proliferation or faster rate of cell proliferation than non-demineralized or fully demineralized cancellous bone matrices, when the cells, e.g., stem cells, are seeded on the bone matrices. Also, the demineralized cancellous bone matrices described herein may exhibit more cell proliferation as compared to non-demineralized or fully demineralized cancellous bone matrices in about 1 day to about 4 weeks, about 1 day to about 2 weeks, about 1 day to about 1 week, about 1 day to about 3 days after seeding of the cells. Furthermore, the demineralized cancellous bone matrices described herein may exhibit more cell proliferation as compared to non-demineralized or fully demineralized cancellous bone matrices in about 1 day, about 2 days, about 3 days, about 4 days, about 5 days, about 7 days (1 week), about 10 days, about 2 weeks, about 3 weeks, or about 4 weeks after seeding of the cells. In one embodiment, the demineralized cancellous bone matrices promote proliferation of mesenchymal stem cells.

### Method of Preparing Demineralized Cancellous Bone Matrices

The cancellous bone used to make the demineralized cancellous bone matrices can be obtained from long bones or other bones. The long bones or other bones are first processed into cross-sections of varying thicknesses. In some embodiments, the thicknesses can range from about 1 mm to about 100 mm or about 5 mm to about 60 mm.

In certain embodiments, the cross-sections of cancellous bone are, for example, greater than or equal to: about 1 mm thick, about 5 mm thick, about 10 mm thick, about 15 mm thick, about 20 mm thick, about 25 mm thick, about 30 mm thick, about 35 mm thick, about 40 mm thick, about 45 mm thick, or about 50 mm thick.

After processing the long bones into cross-sections, the cross-sections of bone are cut or milled into cancellous bone matrices having the desired shape and dimensions. The cutting or milling of the bone can be achieved by using a mechanical press, a punching device, a cross-cutting device, or any other art-known device suitable for creating shaped bone matrices. Furthermore, the bone can be cleaned before and/or after it is milled. As discussed above, the bone can be cleaned using, for example, hydrogen peroxide, detergent and/or ethanol. The bone can be demineralized before or after milling.

In certain embodiments, cancellous bone matrices are cut to obtain the size and shape of the cancellous bone matrices before demineralization of the cancellous bone matrices to obtain the residual calcium levels described above. In such embodiments, the bone can be cleaned before and/or after it is cut. In some embodiments, cancellous bone matrices cut from a bone, for example from condyles, may be exposed to a bone cleaning agent, such as but not limited to a detergent (e.g., 1% polysorbate solution), an antibiotic (e.g., gentamicin), hydrogen peroxide, water, or alcohol (e.g., ethanol) or combinations thereof. The demineralized cancellous bone matrices may also be exposed to a cleaning agent one or more times during the manufacturing process. The cancellous bone matrix may be exposed to one or more bone cleaning agent before and/or after demineralization. The demineralized cancellous bone matrices may be agitated, sonicated, stirred (e.g., mechanically stirred), rinsed, or soaked in one or more bone cleaning agent. Demineralization of the cancellous bone may be conducted in HCl of a certain concentration such as, but not limited to, about 0.2N to about 1.0N, about 0.2N to about 0.5N, or about 0.3N to about 0.6N. The demineralization can occur for a time period such as, but not limited, to about 2 minutes to about 15 minutes or about 5 minutes to about 10 minutes. For example, demineralization can be conducted in about 0.2N to about 0.5N HCl for about 5 minutes to about 10 minutes, about 0.2N to about 0.4N HCl for about 7 minutes to about 11 minutes, or about 0.5N to about 0.7N HCl for about 3 minutes to about 7 minutes.

In certain embodiments, the cancellous bone matrices may be demineralized such that they remain rigid. Any demineralization conditions (such as acid concentration and the time period of demineralization) that result in demineralized cancellous bone matrices that are rigid and comprise residual weight of calcium, such as described above, can be used.

Following demineralization, physiological pH levels of the demineralized cancellous bone matrices can be restored by soaking the demineralized bone in a buffered salt solution. The demineralized cancellous bone matrices can then be lyophilized.

Following lyophilization, the dehydrated, freeze-dried demineralized cancellous bone matrices may be re-hydrated using a saline or a buffered salt solution, e.g., phosphate buffered saline (PBS), and/or a suitable carrier solution, such as, but not limited to, sodium hyaluronate, such as that discussed above. If a carrier is added, excess carrier solution can be removed from the demineralized cancellous bone matrices, and the demineralized cancellous bone matrices can be loaded into a delivery container that is designed to facilitate minimally invasive delivery of the demineralized cancellous bone matrices.

The process of making demineralized cancellous bone matrices may further include combining one or more additional component(s) with the demineralized cancellous bone matrices. The additional component(s) include, but not limited to, bone marrow aspirate, blood, platelet rich plasma, platelet rich plasma matrix, one or more types of cells (stem cells, epithelial cells, fibroblasts, osteoblasts and osteoclasts), chemotactic factors, or growth factors, cortical bone, cytokines, connective tissue or combinations thereof. The one or more additional component(s) may be seeded, coated, injected, infiltrated or impregnated on or into the demineralized cancellous bone matrices. For example, stem cells, e.g. mesenchymal stem cells, other pluripotent cells, osteoblasts, progenitor cells, chondrocytes, and nucleus pulposus cells, may be seeded, coated, injected, infiltrated or impregnated on or into demineralized cancellous bone matrices. One or more additional component(s) may be added to demineralized cancellous bone matrices before or after lyophilization.

In certain embodiments, one or more additional component(s) may be added to the demineralized cancellous bone matrices about 30 minutes to about 3 months, about 30 minutes to about 1 month, about 30 minutes to about 2 weeks, about 30 minutes to about 1 week, about 30 minutes to about 24 hours, or about 30 minutes to about 6 hours prior to insertion of the demineralized cancellous bone matrices into a patient. In some embodiments, one or more additional component(s) may be added to the demineralized cancellous bone matrices less than or equal to about 30 minutes, about 1 hour, about 2 hours, about 3 hours, about 6 hours, about 12 hours, about 24 hours (or about 1 day), about 2 days, about 3 days, about one week, about 2 weeks, about 3 weeks, about 1 month or about 3 months prior to insertion of the demineralized cancellous bone matrices into a patient. Furthermore, in some embodiments, one or more additional component(s) may be added to demineralized cancellous bone matrices about 30 minutes to about 3 months, about 30 minutes to about 1 month, about 30 minutes to about 2 weeks, about 30 minutes to about 1 week, about 30 minutes to about 24 hours, or about 30 minutes to about 6 hours prior to insertion of the demineralized cancellous bone matrices into a void or defect in a bone of a patient. Moreover, in some embodiments, one or more additional component(s) may be added to demineralized cancellous bone matrices less than or equal to about 30 minutes, about 1 hour, about 2 hours, about 3 hours, about 6 hours, about 12 hours, about 24 hours (or about 1 day), about 2 days, about 3 days, about one week, about 2 weeks, about 3 weeks, about 1 month or about 3 months prior to insertion of the demineralized cancellous bone matrices into a void or defect in a bone of a patient.

The process of making the demineralized cancellous bone matrices can further include a step of modifying the amount of cortical bone, growth factors, cytokines and/or connective tissue in the demineralized cancellous bone matrices. For instance, cortical bone, growth factors, cytokines and/or connective tissue can be removed from the demineralized cancellous bone matrices. The step of removing cortical bone, growth factors, cytokines and/or connective tissue from the demineralized cancellous bone matrix can be performed before or after demineralization step. The step of removing cortical bone, growth factors, cytokines and/or connective tissue from the demineralized cancellous bone matrix can be performed before or after the cutting or milling step. In one embodiment, the cortical bone, growth factors, cytokines and/or connective tissue is removed prior to cutting and/or demineralization of the cancellous bone matrices. In one embodiment, cortical bone, the growth factors cytokines and/or connective tissue can be removed after demineralization of the cancellous bone matrices.

The process of making the demineralized cancellous bone matrices can further include a step of testing blood adsorption or blood wicking capability of the demineralized cancellous bone matrices. The step of testing blood adsorption or blood wicking capability of the demineralized cancellous bone matrices may be performed after cutting and demineralization of the cancellous bone matrices. The step of testing blood adsorption or blood wicking capability of the demineralized cancellous bone matrices may be performed before or after lyophilization of the demineralized cancellous bone matrices. Methods of testing blood adsorption capability of the demineralized cancellous bone matrices can include placing a quantity of blood on top of a demineralized cancellous bone matrix. The amount of blood adsorbed by the demineralized cancellous bone matrix is determined by methods such as visual methods. Methods of testing blood wicking capability of the demineralized cancellous bone matrices can include placing a demineralized cancellous bone matrix on top of a quantity of blood. The flow of blood against the force of gravity in the demineralized cancellous bone matrix can be observed. Also, the time required to adsorb/wick the blood can be observed to determine the adsorption and/or wicking capability.

### Implants Comprising Demineralized Cancellous Bone Matrices

The demineralized cancellous bone matrices as described herein may generally be delivered to and implanted in a bone having a void or defect located in the body of a patient for treating the patient, such as repairing defects in bone. Also, as discussed below, the demineralized cancellous bone matrices are designed to be delivered through a minimally invasive route into a cavity in a patient.

The demineralized cancellous bone matrices can be used to treat various bones. These bones include without limitation long bones, (e.g., a femur, tibia, fibula, humerus), bones of the spine, pelvic bones, the skull and bones of the extremities. In certain embodiments, as discussed further below, the demineralized cancellous bone matrices may be used to treat defects of the spine, such as ones in a vertebral body or in an interbody space between two vertebrae.

The demineralized cancellous bone matrices can be designed to conform to a bone void or defect, for example, such that bone void or defect is completely filled, substantially filled or partially filled upon implantation. The demineralized cancellous bone matrices can be designed to conform to a bone void or defect such that they fill the bone void or defect with tight apposition against host bone. The demineralized cancellous bone matrices may provide mechanical support, induce or accelerate new bone growth in the bone void or defect, and/or promote remodeling of the bone void or defect site. The demineralized cancellous bone matrices may be osteoinductive, osteogenic and/or osteoconductive.

In one embodiment, the demineralized cancellous bone matrices described herein may be used to fill a bone void or defect in a bone, such as one resulting from a traumatic injury or bone tumor. A bony tumor resulting in a bone void or defect can be benign or malignant (i.e., bone cancer), and may be a primary tumor that originates in the bone or a secondary tumor which originate elsewhere. Examples of benign bone tumors include, but not limited to, osteoma, osteoid osteoma, osteochondroma, osteoblastoma, enchondroma, giant cell tumor of bone, aneurismal bone cyst, and fibruous dysplasia of bone. Malignant primary bone tumors include, but not limited to, osteosarcoma, chondrosarcoma, fibrosarcoma, and other sarcoma types.

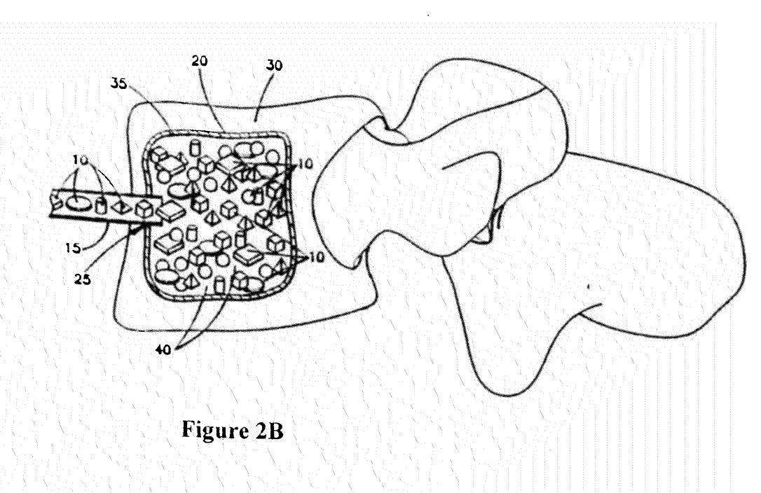

In some embodiments, the method of treating bone having a void or defect can comprise inserting into a void or defect at least one demineralized cancellous bone matrix. FIG. 2A shows an implant comprising a plurality of demineralized cancellous bone matrices being inserted into a void in a femur.

The size and/or volume of a bone void or defect can be estimated as described below, and the demineralized cancellous bone matrices of the size and shape required to fill the void or defect can be selected. The insertion of the implant comprising at least demineralized cancellous bone matrix may be mediated by a delivery container, for example by a cannula. A delivery container may be inserted into the opening of the bone void or defect. An implant as described herein can be passed into the bone void or defect through the delivery container. In certain embodiments, in order to facilitate delivery, the demineralized cancellous bone matrices may be loaded into the delivery container prior to the time of surgery. In alternate embodiment, the demineralized cancellous bone matrices may be loaded into the delivery container during the time of surgery. The demineralized cancellous bone matrices described herein are designed so that it is easy for a surgeon or other assisting persons to load the demineralized cancellous bone matrices into such delivery containers.

As shown in FIG. 2A, the demineralized cancellous bone matrices 10 can be contained in a delivery container 15 for delivery into the bone void or defect 20. The delivery container 15 is inserted into the bone void or defect 20, and the demineralized cancellous bone matrices 10 are passed into the bone void or defect 20 until the desired amount of demineralized cancellous bone matrices 10 is placed into the bone void or defect 20.

In other embodiments, the one or more demineralized cancellous bone matrix are inserted directly into the cavity in the bone of a patient. The one or more demineralized cancellous bone matrix may be inserted manually or using a surgical instrument, for example using forceps. The implant may be shaped and sized to fill the bone void or defect upon insertion. For example, an implant comprising one or more demineralized cancellous bone matrix of an irregular polygonal shape or a wedge shape may be inserted using surgical forceps to fill the wedge-shaped void or cavity in a bone of a patient. In some embodiments, there is a void or free space between the one or more demineralized cancellous bone matrix and the surrounding tissue of the bone void or defect. In yet other embodiments, where more than one demineralized cancellous bone matrix is used, there is a void or free space between the demineralized cancellous bone matrices in the bone void or defect. In certain embodiments, there is no void or free space or substantially no void or free space between the one or more demineralized cancellous bone matrices and the surrounding tissue of the bone void or defect and/or between the plurality of demineralized cancellous bone matrices in the bone void or defect.

In another embodiment, the demineralized cancellous bone matrices described herein may be used to repair a fractured or collapsed vertebral body, such as one resulting from a vertebral compression or burst fracture. In some embodiments, the method of treating a vertebral body compression or burst fracture in a patient can comprise the steps of accessing a target vertebral body of the patient, creating a cavity having a volume within the vertebral body, and implanting into the cavity an implant comprising at least one demineralized cancellous bone matrices. FIG. 2B shows an implant comprising at least one demineralized cancellous bone matrix being implanted into a vertebral body.

More specifically, first, a target vertebral body 30 in a patient is accessed by positioning a guide wire either into the pedicle or parallel to the pedicle under fluoroscopic guidance. Subsequently, a cannula is placed over the guide wire that serves as an access portal. After the cannula is secured to the vertebral body, the guide wire is removed and cavity creation tools are utilized in order to create space for the one or more demineralized cancellous bone matrices and/or the implantable container for the implant.

Next, at least one cavity 20 having a volume is created within the target vertebral body 30. Although only one cavity is shown in FIG. 2B, in other embodiments, there may be more than one cavity. The cavity 20 has at least one opening 25. The opening 25 of the cavity 20 may be created by, for example, removal of bony vertebral material by, e.g., reaming, drilling, or scraping, followed by evacuation of the bone particles. The cavity may also be enlarged by the expansion of the expandable container under pressurized filling.

After the formation of a cavity 20 in the vertebral body 30, the resulting cavity can be sized, e.g., as described in United States Publication No. 2008/0027546 to Semler et al., which is incorporated herein by reference in its entirety. The sizing step may consist of inserting an inflatable balloon in the cavity and filling the cavity with radio-contrast fluid to a specific pressure between about 30 psi to about 60 psi such that the cavity is visible under fluoroscopy. This step allows visualization of the cavity created and also provides a measurement of the cavity volume, which is used to determine the amount of demineralized cancellous bone matrix needed.

Next, an implant as described herein, comprising at least one demineralized cancellous bone matrix 10, is inserted into the cavity. As shown in FIG. 2B, the demineralized cancellous bone matrices 10 can be contained in a delivery container 15, such as that shown in FIG. 1B, for delivery into the cavity 20. In certain embodiments, in order to facilitate delivery, the demineralized cancellous bone matrices may be loaded into the delivery container prior to the time of surgery. In alternate embodiments, the demineralized cancellous bone matrices may be loaded into the delivery container during the time of surgery. The demineralized cancellous bone matrices described herein are designed so that it is easy for a surgeon or other assisting persons to load the demineralized cancellous bone matrices into such delivery containers.

In some embodiments, as shown in FIG. 2B, the delivery container 15 is inserted into the opening 25 of the cavity 20 and the one or more demineralized cancellous bone matrix 10 is passed into the cavity 20 located in the vertebral body 30. In FIG. 2B, the demineralized cancellous bone matrices 10 are passed into the cavity 20 until the desired amount of demineralized cancellous bone matrices 10 is placed into the cavity 20.

In some embodiments, the one or more demineralized cancellous bone matrix is inserted directly into the cavity, such as vertebral body cavity, in the patient. In other embodiments, such as that shown in FIG. 2B, an implantable container 35 is inserted into the cavity 20 through the opening 25 before the demineralized cancellous bone matrices 10 of the implant are inserted into the cavity 20. The implantable container 35 is initially empty and in a collapsed state such that it can be passed through the opening 25 of the cavity 20. The demineralized cancellous bone matrices are then inserted into the implantable container 35 that is already located within the cavity 20. Afterwards, the implantable container 35 and/or cavity 20 may be closed or sealed.

In some embodiments, the implantable container is expandable. The implantable container may be expanded in the cavity before the demineralized cancellous bone matrices are inserted therein or expanded by the process of inserting the implant into the container. The implantable container may be made from synthetic materials such as, but not limited to, polyester, or from biological materials such as, but not limited to, allograft bone, dermis, or fascia, hyaluronic acid, collagen, or other structural protein.

In some embodiments, the implantable container is porous and comprises, e.g., a mesh, such as a woven fabric mesh. The implantable container can be a mesh bag. In these configurations, the pores of the implantable container will allow bone to grow into the implant site. The pores of the implantable container may also serve to allow the transfer of fluid and materials, such as cells, between the surrounding tissue and the implant site. Also, the implantable container may have pore sizes that are sufficiently small such that the demineralized cancellous bone matrices do not readily fall through the pores. In particular embodiments, the implantable container may also possess radiopaque properties such that it is visible during implantation.

When the demineralized cancellous bone matrices 10 are implanted into the cavity 20, or when the demineralized cancellous bone matrices 10 are implanted into the implantable container 35 within the cavity 20 (as shown in FIG. 2B), there can be void spaces 40 between the demineralized cancellous bone matrices 10. The void spaces facilitate the transfer of fluid and materials between the surrounding tissue and the implant site, which may facilitate cellular penetration and graft incorporation. In yet other embodiments, there are no void spaces between the demineralized cancellous bone matrices of the implant. In yet another embodiment, there are no void spaces between the one or more demineralized cancellous bone matrix and the surrounding tissue of the bone void, bone defect or cavity.

In certain embodiments, the demineralized cancellous bone matrices occupy less than 100% of the volume of the bone void, bone defect, cavity or implantable container. For example, the demineralized cancellous bone matrices may occupy about 25% to about 99%, about 75% to about 95%, about 75% to about 99% or about 80% to about 90% of the volume of the bone void, bone defect, cavity or implantable container. In some embodiments, the demineralized cancellous bone matrices may occupy greater than or equal to: about 99%, about 98%, about 97%, about 96%, about 95%, about 94%, about 93%, about 92%, about 91%, about 90%, about 85%, about 80%, about 75%, about 70%, about 65%, about 60%, about 55%, about 50%, or about 25% of the volume of the bone void, bone defect, cavity or implantable container when implanted therein. In other embodiments, the demineralized cancellous bone matrices may occupy less than or equal to: about 99%, about 98%, about 97%, about 96%, about 95%, about 94%, about 93%, about 92%, about 91%, about 90%, about 85%, about 80%, about 75%, about 70%, about 65%, about 60%, about 55%, about 50%, or about 25% of the volume of the bone void, bone defect, cavity or implantable container when implanted therein.

The percentage of the volume of the bone void, bone defect, cavity or implantable container occupied by the demineralized cancellous bone matrices may be directly related to the size and shape of the demineralized cancellous bone matrices. For instance, in certain embodiments wherein the demineralized cancellous bone matrices are larger in size, this may create larger void spaces in between each demineralized cancellous bone matrix, leading to a decreased percentage of the volume of the bone void, bone defect, cavity or implantable container occupied by the demineralized cancellous bone matrices. Conversely, certain embodiments, wherein the demineralized cancellous bone matrices are smaller in size, may allow for smaller void spaces in between each demineralized cancellous bone matrix, leading to an increased percentage of the volume of the bone void, bone defect, cavity or implantable container occupied by the demineralized cancellous bone matrices. Moreover, in certain embodiments wherein the bone matrices have a certain shape, such as a spherical or cuboidal shape, this may also create larger void spaces in between each demineralized cancellous bone matrix, leading to a decreased percentage of the volume of the bone void, bone defect, cavity or implantable container occupied by the demineralized cancellous bone matrices.

In some embodiments, when demineralized cancellous bone matrices are implanted in the bone void, bone defect, cavity or implantable container, the packing or bulk density of the demineralized cancellous bone matrices in the bone void, defect, cavity or implantable container can be about 0.01 g/cc to about 5.00 g/cc, about 0.10 g/cc to about 2.00 g/cc, about 0.20 g/cc to about 1.40 g/cc, about 0.40 g/cc to about 1.00 g/cc, about 0.50 g/cc to about 0.80 g/cc, or about 0.50 g/cc to about 1.00 g/cc based on dry weight of the bone. In particular embodiments, the demineralized cancellous bone matrices described herein can have a packing density of about 0.50 g/cc to about 0.80 g/cc based on dry weight of bone. In other embodiments, the demineralized cancellous bone matrices described herein have a packing density of about 0.60 g/cc to about 0.80 g/cc based on dry weight of the bone matrices or implant material.

The amount of one or more demineralized cancellous bone matrices that are implanted into the implant site may be varied for the specific size of the bone void, bone defect or cavity. In certain embodiments, the volume of the one or more demineralized cancellous bone matrices that are implanted can be greater than that of the initial volume of the cavity. For example, the one or more demineralized cancellous bone matrix may provide a degree of restoration of vertebral body shape or height in a collapsed or fractured vertebral body. The one or more demineralized cancellous bone matrices described herein may also possess mechanical properties that withstand the compressive loads, e.g., compressive loads in the spine when implanted into the cavity of the patient.

After the demineralized cancellous bone matrices are inserted into the bone void, bone defect, cavity or implantable container within the cavity, the opening of the bone void, bone defect, cavity and/or implantable container may be left open. Alternatively, after the one or more, demineralized cancellous bone matrix is implanted, the opening to the bone void, bone defect, cavity and/or implantable container may be sealed with a material including, but not limited to, a biocompatible sealant. Materials that may be used as biocompatible sealants include, without limitation, an allograft bone plug, a ceramic, polymeric or metallic plug, and fibrin glue.

Moreover, in certain embodiments, the demineralized cancellous bone matrices can be used to treat spinal discs located between adjacent vertebrae. In some embodiments, the demineralized cancellous bone matrices can be used to create a fusion between two adjacent vertebral bodies. This type of procedure can be used to address conditions associated with mild to severe disc degeneration or other spinal deformities. In one embodiment, the fusion procedure may comprise forming at least one cavity, having a volume and an opening, between two adjacent vertebral bodies. An implant comprising at least one or more demineralized cancellous bone matrices described herein can then be implanted into the cavity.

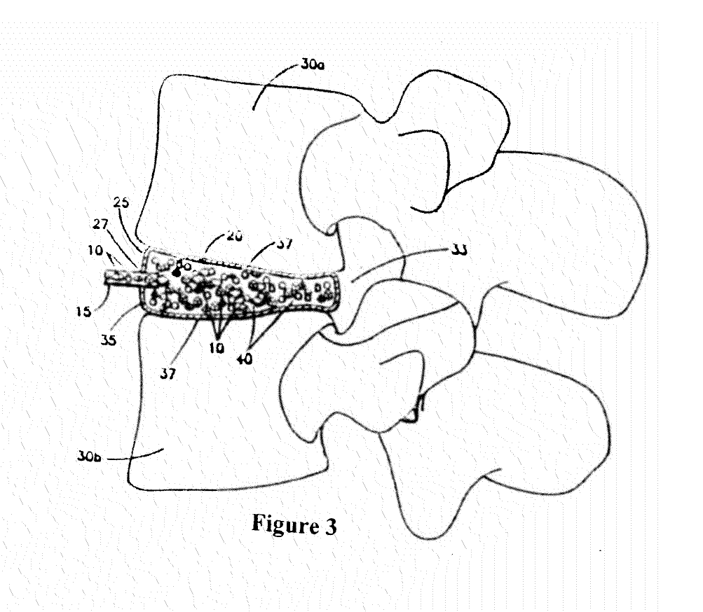

FIG. 3 shows a plurality of demineralized cancellous bone matrices being implanted in the space between two vertebral bodies. In this embodiment, a targeted intervertebral disc space 33 is accessed. The disc space in a patient can be accessed by positioning a guide wire either into the disc from either an anterior, posterior, posterolateral, anterolateral, or lateral approach to the spine under fluoroscopic guidance. Subsequently, a cannula is placed over the guide wire that serves as an access portal. After the cannula is secured to the disc, the guide wire is removed and cavity creation tools are utilized in order to create space for the demineralized cancellous bone matrices and/or the expandable container for the implant.

Thereafter, all or a portion of the intervertebral disc is removed to create a cavity 20, having a volume, between the two adjacent intervertebral bodies 30a and 30b. The cavity may be created by removing at least a portion of the intervertebral disc, e.g., by microdiscectomy, minimally invasive nucleotomy, or by, e.g., reaming, drilling, gouging or scraping followed by evacuation of the disc fragments. An opening to the cavity may be created during the formation of the cavity as described herein.

After the cavity is created, the endplates 37 of the vertebral bodies 30a and 30b can be decorticated to access bleeding bone. The endplates 37 can be decorticated by gouging, scraping, cutting or piercing tools. Also, the cavity can be sized before the demineralized cancellous bone matrices are implanted by, for example, the methods discussed above.

An implantable container 35, such as that discussed herein, can be used. As shown in FIG. 3, an implantable container 35 is inserted into the cavity 20 before the demineralized cancellous bone matrices 10 are inserted into the cavity 20. The implantable container 35 may be expanded before the demineralized cancellous bone matrices are placed into the implantable container. A delivery container 15 is inserted into the opening 27 of the implantable container 35 and the demineralized cancellous bone matrices 10 are passed into the implantable container 35 in the cavity 20. If an implantable container is not used, a delivery container 15 can be inserted into the opening of the cavity 25 and the demineralized cancellous bone matrices 10 can be passed into the cavity 20. The demineralized cancellous bone matrices 10 are passed into the container 35 or cavity 20 until the desired amount of demineralized cancellous bone matrices 10 is placed into the implantable container 35 or cavity 20. After the demineralized cancellous bone matrices have been implanted, the implantable container 35 and/or cavity 20 may be closed or sealed.

As illustrated in FIG. 3, when the demineralized cancellous bone matrices 10 are implanted into the cavity 20 between the vertebral bodies 30a and 30b or implantable container 35 within the cavity 20, there may be void spaces 40 between the demineralized cancellous bone matrices 10. As discussed above, the void spaces facilitate the transfer of fluid and materials between the surrounding tissue and the implant site, which may facilitate cellular penetration and graft incorporation. There also may be void spaces between at least one demineralized cancellous bone matrices and the surrounding tissue of the cavity or between at least one demineralized cancellous bone matrices and the implantable container. In other embodiments, there may be no or substantially no void spaces between at least one demineralized cancellous bone matrix and the surrounding tissue of the cavity or implantable container, or between the plurality of the demineralized cancellous bone matrices of the implant in the cavity.

In addition, in certain embodiments, as described above, the demineralized cancellous bone matrices may occupy a certain percentage of the volume of the cavity or implantable container. Also, in some embodiments, when the implant is implanted in the cavity or implantable container, the packing or bulk density of the demineralized cancellous bone matrices can be a certain value.

As with the demineralized cancellous bone matrices used to address vertebral fractures discussed previously, the amount of demineralized cancellous bone matrices that is implanted into the implant site may be varied. For instance, the volume of the demineralized cancellous bone matrices can be greater than that of the initial volume of the cavity so that they may provide mechanical properties that withstand the compressive loads in the spine when implanted into the cavity of the patient.

Additionally, in certain embodiments, the implants described herein may be used to repair or replace a part or all of a spinal disc without fusion of the vertebrae. Also, the implant may be used to augment the spinal disc or restore the height of the spinal disc. For example, the implant may be used to replace all or part of the nucleus pulposus of the spinal disc. In these embodiments, an opening is made in the spinal disc. All or part of the nucleus pulposus is removed to create a cavity in the spinal disc that is located between two adjacent vertebrae. The methods described above in creating a cavity for spinal fusion may be used to remove the nucleus pulposus and create the cavity in the spinal disc. The implant is then inserted into the cavity in the spinal disc. Moreover, an implantable container may be used as described above. The methods described for inserting the demineralized cancellous bone matrices and implantable container in connection with spinal fusions can be used to insert the demineralized cancellous bone matrices and implantable container into the cavity in the spinal disc.

Furthermore, in certain embodiments where the demineralized cancellous bone matrices described herein are used to repair or replace a part of a spinal disc without fusion of the vertebrae, at least some or all of the demineralized cancellous bone matrices can be non-osteoinductive. The demineralized cancellous bone matrices can be rendered non-osteoinductive by, for example, exposing the demineralized cancellous bone to hydrogen peroxide for a certain amount of time during the preparation of the bone used in the implants. In one embodiment, after the demineralized cancellous bone is demineralized, it can be exposed to hydrogen peroxide for at least 1 hour. In other embodiments, the demineralized cancellous bone can be rendered non-osteoinductive by exposing the demineralized cancellous bone to heat, radiation or chemicals.

The description contained herein is for purposes of illustration and not for purposes of limitation. The methods and constructs described herein can comprise any feature described herein either alone or in combination with any other feature(s) described herein. Changes and modifications may be made to the embodiments of the description. Furthermore, obvious changes, modifications or variations will occur to those skilled in the art. Also, all references cited above are incorporated herein, in their entirety, for all purposes related to this disclosure.

The following illustrative examples are set forth to assist in understanding the methods and constructs described herein and do not limit the claimed methods and constructs.

### EXAMPLES

### Example 1

### Preparation of Demineralized Cancellous Bone Matrix

Cancellous bone matrix was obtained by cutting cancellous bone from condyles into cancellous bone matrices of various shapes. Lipids were removed from the cancellous bone matrices using 1% polysorbate solution. The cancellous bone matrices were then decontaminated in a gentamicin solution, rinsed with water, and cleaned using hydrogen peroxide. The cancellous bone matrices were subsequently demineralized in 0.6N HCl for 5 minutes or in 0.3N HCl for 9 minutes, to reach a residual calcium level between about 6% by weight and about 15% by weight. The demineralized cancellous bone matrices were then further cleaned and lyophilized to a residual moisture content of less than 6 wt %.

### Example 2

### Preparation of Fully Demineralized Cancellous Bone Matrix

Cancellous bone matrix was obtained by cutting cancellous bone from condyles into cancellous bone matrices of various shapes. Lipids were removed from the cancellous bone matrices using 1% polysorbate solution. The cancellous bone matrices were then decontaminated in a gentamicin solution, rinsed with water, and cleaned using hydrogen peroxide. The cancellous bone matrices were subsequently demineralized in 0.6N HCl for 24 hours, to reach a residual calcium level of less than 0.5% by weight. The fully demineralized cancellous bone matrices were then further cleaned and lyophilized to a residual moisture content of less than 6 wt %.

### Example 3

### Preparation of Non-Demineralized Cancellous Bone Matrix

Cancellous bone matrix was obtained by cutting cancellous bone from condyles into cancellous bone matrices of various shapes. Lipids were removed from the cancellous bone matrices using 1% polysorbate solution. The cancellous bone matrices were then decontaminated in a gentamicin solution, rinsed with water, and cleaned using hydrogen peroxide. The cancellous bone matrices, which were not demineralized, were then further cleaned and lyophilized to a residual moisture content of less than 6 wt %.

### Example 4

### Blood Adsorption and Blood Wicking by Non-Demineralized, Demineralized and Fully Demineralized Cancellous Bone

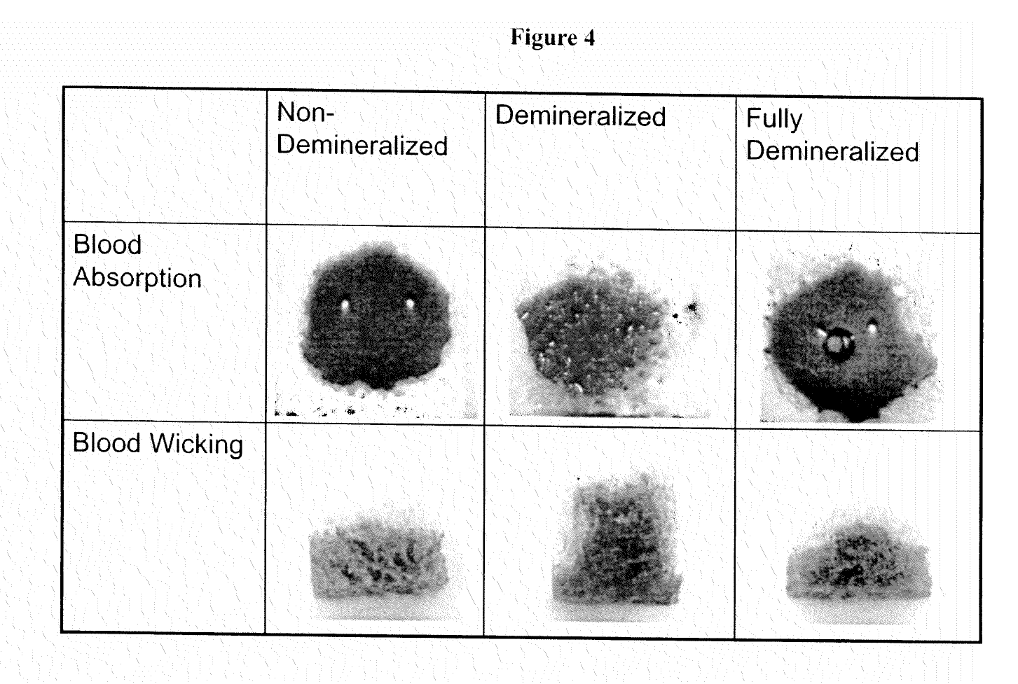

To determine blood adsorption ability of the cancellous bone matrices of Examples 1 through 3 above, drops of blood were placed on (1) a demineralized cancellous bone matrix obtained by the process described in Example 1; (2) a fully demineralized cancellous bone matrix obtained by the process described in Example 2; and (3) a non-demineralized cancellous bone matrix obtained by the process described in Example 3. The adsorption of the blood by each of these three types of cancellous bone matrices was visually observed after placing drops of blood on top of the cancellous bone matrices. FIG. 4 shows the blood adsorbed by the three types of cancellous bone matrices. The drops of blood were adsorbed quickly (less than 10 seconds) by the demineralized cancellous bone matrix. The non-demineralized cancellous bone matrix adsorbed the blood in 2 minutes. The fully demineralized cancellous bone matrix did not show signs of adsorption of the blood until after 5 minutes.

The blood wicking ability of the demineralized, fully demineralized and non-demineralized cancellous bone matrices prepared by the process described in Examples 1 to 3, respectively, was determined. Samples of each of the three types of cancellous bone matrices were placed on top of drops of blood for 5 minutes. FIG. 4 shows the blood wicking capacity of the three types of cancellous bone matrices. The demineralized cancellous bone matrices displayed superior blood wicking ability compared to the fully demineralized or non-demineralized cancellous bone matrices, as demonstrated by their ability to draw blood vertically up the height of the bone matrix by capillary action. Specifically, the demineralized cancellous bone matrices were able to pull blood further up the height of the bone matrix from the point of contact with the blood compared to the fully demineralized or non-demineralized cancellous bone matrices.

### Example 5

### Cell Adhesion and Proliferation Studies Using Bone Marrow-Derived MSCs

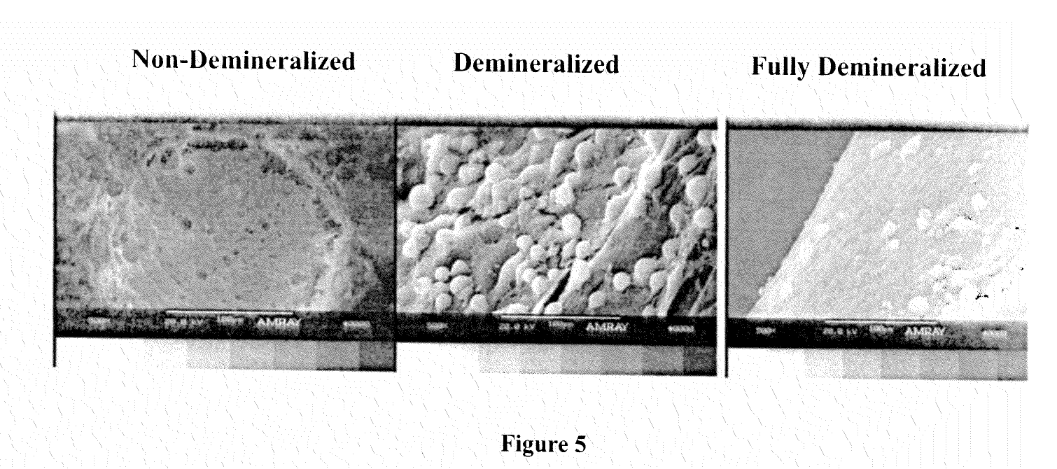

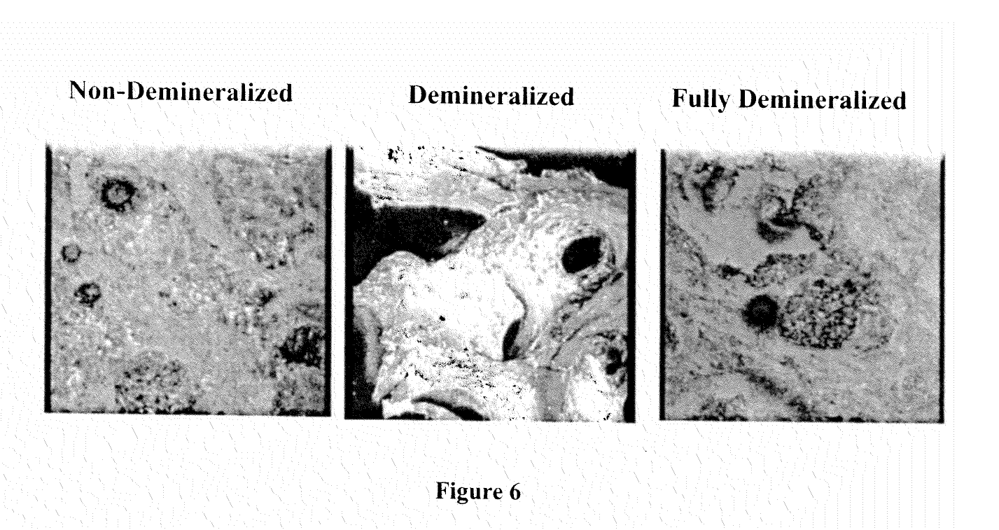

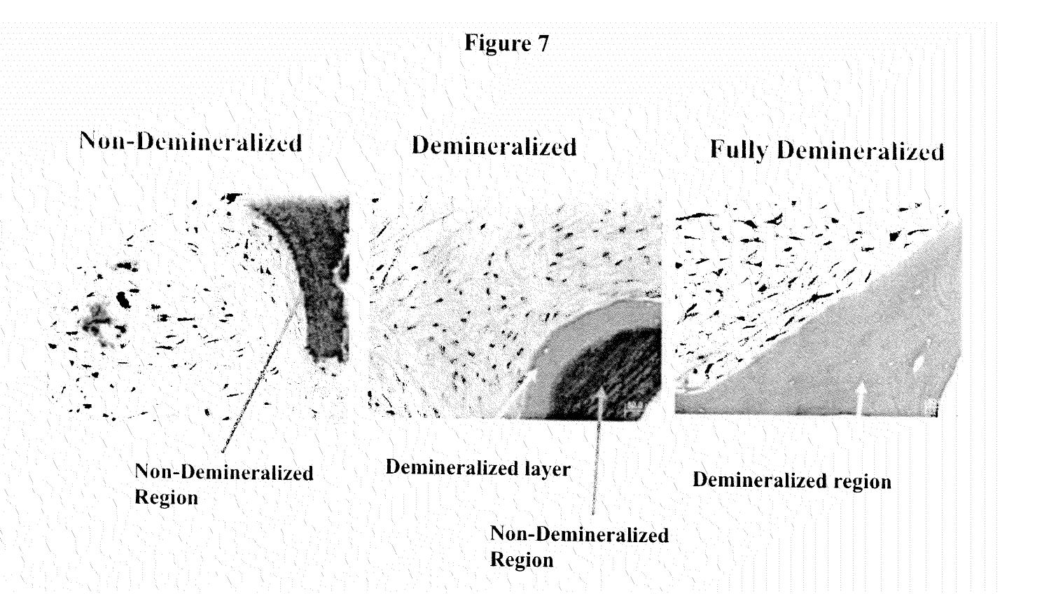

Human bone marrow-derived mesenchymal stem cells (“MSC”) were obtained from Lonza. MSC were cultured in expansion basal medium (MSCGM BulleKit from Lonza Walkersville, Inc.). Demineralized, fully demineralized and non-demineralized cancellous bone matrices were prepared as described in Examples 1 to 3 above. MSC were seeded at a density of 2×10⁷ cells/ml onto each type of cancellous bone matrix maintained in osteogenic media (Osteogenic differentiation media BulleKit from Lonza Walkersville, Inc.). Cell adhesion and cell proliferation of MSC on the cancellous bone matrices were evaluated using scanning electron microscopy (“SEM”) (FIG. 5), multiphoton microscopy (FIG. 6) and histologic analysis (FIG. 7). Samples for SEM and multiphoton microscopy were fixed in 10% and 4% formalin, respectively, prior to microscopy imaging. Samples for histological analysis were fixed in 10% formalin prior to paraffin embedding, sectioning and staining.

Cell adhesion to the cancellous bone matrices was evaluated by scanning electron microscopy at 12 hours after seeding of MSC. FIG. 5 shows that 12 hours after seeding, more MSC attached to the surface of demineralized cancellous bone matrices as compared to the number of cells attached to the surface of fully demineralized and non-demineralized bone matrices.

Cell proliferation of MSC on the cancellous bone matrices was analyzed by multiphoton microscopy at 5 days after seeding of MSC. Live MSC were labeled with CellTracker Green CMFDA (5-chlormethylfluorescein diacetate) (Molecular Probes) prior to seeding of the cells on the cancellous bone matrices (CMFDA is a green fluorescent dye that freely diffuses through the membrane of live cells, and once inside, can react with thiols on proteins and peptides). The cancellous bone matrices were fluorescently labeled with AlexaFluor 633 Carboxylic acid, succinimidyl ester (Molecular Probes) prior to cell seeding. AlexaFluor 633 Carboxylic acid, succinimidyl ester is an amine reactive dye, which allows the red fluorescent label to be conjugated to the amine group on the bone cancellous matrices. The labeled cells were then seeded onto the labeled cancellous bone matrices and incubated in expansion medium. 5-days post-seeding samples were fixed in 4% formalin and imaged. FIG. 6 shows a multiphoton microscopy image of labeled MSC on the surface of demineralized, fully demineralized and non-demineralized cancellous bone matrices. It demonstrates that proliferation of MSC is greater on the surface of the demineralized cancellous bone matrix as compared to the fully demineralized and non-demineralized cancellous bone matrices as evident from the increased green (CMFDA) fluorescence labeling of cells attached to demineralized cancellous bone sample.

FIG. 7 is a histology sample showing hematoxylin and eosin-stained MSC at two weeks after seeding of the cells on the surface of the samples. Representative images were taken using a light microscope at ×100 magnification. Arrows show non-demineralized and demineralized regions of bone. Increased adhesion and proliferation of MSC can be observed on the surface of the demineralized cancellous bone sample as compared to the fully demineralized and non-demineralized cancellous bone samples.

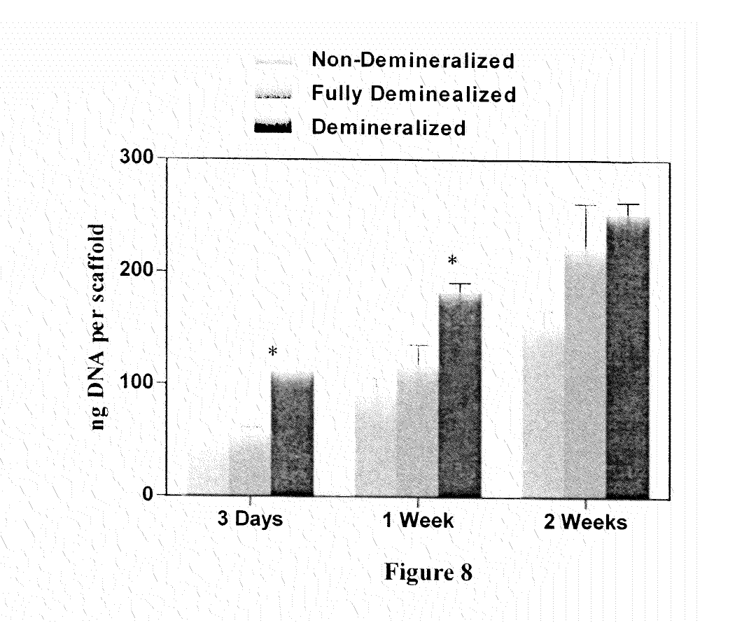

FIG. 8 shows relative levels of cell adhesion and proliferation of MSC on three samples of each of the demineralized, fully demineralized and non-demineralized cancellous bone matrices, by measuring total DNA content of cells attached to the cancellous bone matrices at 3 days, 1 week or 2 weeks after seeding of the cells. At 3 days, 1 week or 2 weeks post-seeding, the samples were rinsed with PBS, lysed and homogenized with proteinase K. The total DNA content was evaluated with a fluorometric DNA quantification (CyQUANT DNA assay, Invitrogen). Sample fluorescence was measured at the excitation and emission wavelengths of 480 nm and 530 nm, respectively. Blank (without MSC) demineralized, fully demineralized and non-demineralized cancellous bone matrices were similarly tested, and had no detectable DNA content value at day “0”. It was determined that DNA content, and thus cell number, of MSC attached to the surface of demineralized cancellous bone matrices was higher than of MSC attached to the surface of fully demineralized or non-demineralized cancellous bone matrices at all tested time points.

### Example 6

### Quantification of BMP-2 in Demineralized Cancellous Matrices

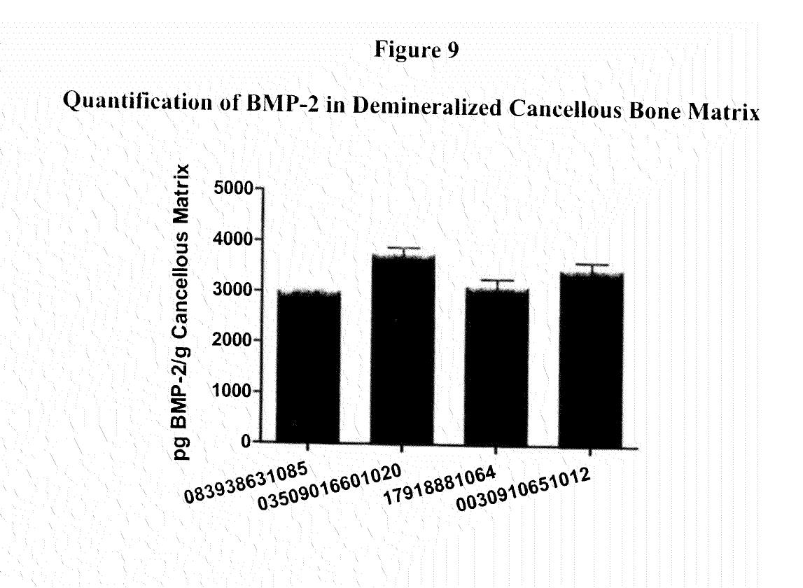

To evaluate osteoinductivity of demineralized cancellous bone matrix with a residual calcium level between about 6 wt % and about 15 wt %, BMP-2 protein content of demineralized cancellous bone matrices prepared as described in Example 1 above was analyzed. Cancellous bone matrices were obtained from four different donors. 50 grams of cancellous bone matrices were weighed, demineralized and lyophilized. 0.25 g of cancellous bone matrices were weighed and extracted using guanidine, followed by dialysis in PBS. The extracts were assayed for BMP-2 protein concentration. BMP-2 protein level was measured using enzyme-linked immunosorbence assay (ELISA) kit (R&D Systems). Absorbance was measured at 450 nm with the correction wavelength set at 540 nm. Results, presented in FIG. 9, are expressed in picogram of BMP-2 protein detected per gram of demineralized cancellous bone matrix.

### Example 7

### Analysis of Alkaline Phosphatase Activity in MSC

Alkaline phosphatase (“ALP”) activity, which is an early marker for osteogenic differentiation, was measured in MSC at 1 week or 2 weeks after seeding of the cells on the three samples of each of the demineralized, fully demineralized and non-demineralized cancellous bone matrices prepared as described in Examples 1 to 3. At 1 week or 2 weeks post-seeding, the samples were rinsed with PBS, lysed and homogenized with 0.2% Triton X-100. The total DNA content of MSC was evaluated with a fluorometric DNA quantification (CyQUANT DNA assay, Invitrogen). Sample fluorescence was measured at the excitation and emission wavelengths of 480 nm and 530 nm, respectively. ALP activity determination was based on conversion of p-nitrophenyl phosphate to p-nitrophenol. A p-nitrophenyl phosphate (pNPP) liquid substrate system was used to analyze the ALP concentration of the MSC samples. 50 μl of cell lysate solution was added to 50 μl of pNPP substrate and incubated at room temperature in dark for 30 minutes. The absorbance was read at 405 nm and normalized to the total DNA content.

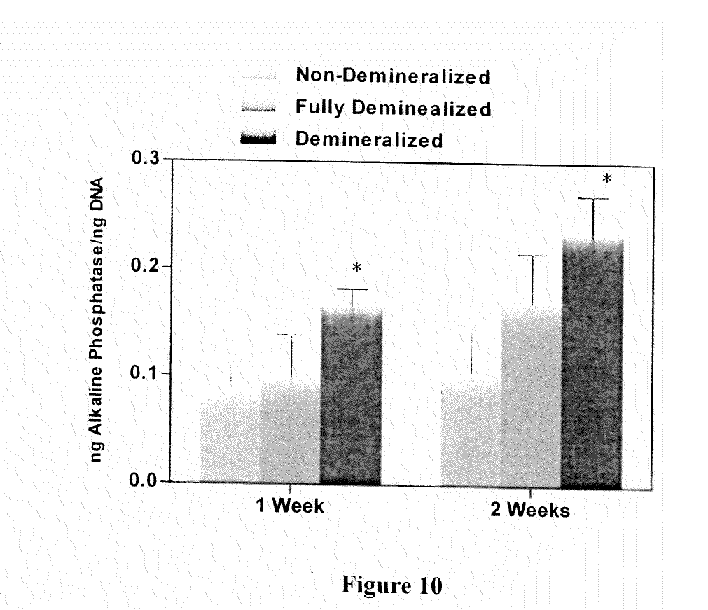

FIG. 10 shows that MSC cultured on demineralized cancellous bone matrices display higher ALP activity as compared to MSC cultured on fully demineralized or non-demineralized cancellous bone matrices.

What is claimed is:

1. A demineralized cancellous bone matrix comprising a cancellous bone matrix that has been demineralized to contain about 5% to about 20% by weight residual calcium, and wherein the demineralized cancellous bone matrix is rigid and has dimensions, wherein one of the dimensions is a length between about 0.5 mm to about 20 mm.

2. The demineralized cancellous bone matrix of claim 1, wherein the demineralized cancellous bone matrix has a length between about 2 mm to about 12 mm.

3. The demineralized cancellous bone matrix of claim 1, wherein the demineralized cancellous bone matrix has a length between about 5 mm to about 10 mm.

4. The demineralized cancellous bone matrix of claim 1, wherein each of the dimensions of the demineralized cancellous bone matrix is individually between about 0.5 mm to about 20 mm.

5. The demineralized cancellous bone matrix of claim 1, wherein each of the dimensions of the demineralized cancellous bone matrix is individually between about 2 mm to about 12 mm.

6. The demineralized cancellous bone matrix of claim 1, wherein each of the dimensions of the demineralized cancellous bone matrix is individually between about 5 mm to about 10 mm.

7. The demineralized cancellous bone matrix of claim 4, wherein the size of the individual dimensions can vary along the dimensions.

8. The demineralized cancellous bone matrix of claim 1, wherein the demineralized cancellous bone matrix has a cuboidal shape.

9. The demineralized cancellous bone matrix of claim 1, wherein at least one surface of the demineralized cancellous bone matrix has an irregular polygonal shape.

10. The demineralized cancellous bone matrix of claim 1, wherein the cancellous bone matrix has been demineralized to contain about 6% to about 15% by weight residual calcium.

11. The demineralized cancellous bone matrix of claim 1, wherein the demineralized cancellous bone matrix is formed from human bone.

12. The demineralized cancellous bone matrix of claim 1, wherein the demineralized cancellous bone matrix is formed from non-human bone.

13. The demineralized cancellous bone matrix of claim 1, which further comprises at least one additional component.

14. The demineralized cancellous bone matrix of claim 13, wherein the at least one additional component comprises bone marrow aspirate, blood, platelet rich plasma, platelet rich plasma matrix, stem cells, epithelial cells, fibroblasts, osteoclasts, osteoblasts, chemotactic factors, growth factors, a carrier, cortical bone, or combinations thereof.

15. The demineralized cancellous bone matrix of claim 14, wherein the carrier comprises saline, phosphate buffered solution, sodium hyaluronate, hyaluronic acid, or combinations thereof.

16. The demineralized cancellous bone matrix of claim 14, wherein the at least one additional component comprises stem cells comprising mesenchymal stem cells.

17. The demineralized cancellous bone matrix of claim 1, wherein the demineralized cancellous bone matrix includes less than or equal to about 10% by weight of cortical bone.

18. The demineralized cancellous bone matrix of claim 1, wherein the demineralized cancellous bone matrix includes less than or equal to about 5% by weight of growth factors, or less than or equal to about 5% by weight of connective tissue.

19. An implant comprising at least one demineralized cancellous bone matrix, wherein the at least one demineralized cancellous bone matrix comprises a cancellous bone matrix that has been demineralized to contain about 5% to about 20% by weight residual calcium, and wherein the at least one demineralized cancellous bone matrix is rigid and has dimensions, wherein one of the dimensions is a length between about 0.5 mm to about 20 mM.

20. The implant of claim 19, wherein the at least one demineralized cancellous bone matrix has a length between about 2 mm to about 12 mm.

21. The implant of claim 19, wherein the at least one demineralized cancellous bone matrix has a length between about 5 mm to about 10 mm.

22. The implant of claim 19, wherein each of the dimensions of the demineralized cancellous bone matrix is individually between about 0.5 mm to about 20 mm.

23. The implant of claim 19, wherein each of the dimensions of the demineralized cancellous bone matrix is individually between about 2 mm to about 12 mm.

24. The implant of claim 19, wherein each of the dimensions of the demineralized cancellous bone matrix is individually between about 5 mm to about 10 mm.

25. The implant of claim 22, wherein the size of the individual dimensions can vary along the dimensions.

26. The implant of claim 19, wherein the at least one demineralized cancellous bone matrix has a cuboidal shape.

27. The implant of claim 19, wherein at least one surface of the at least one demineralized cancellous bone matrix has an irregular polygonal shape.

28. The implant of claim 19, wherein the cancellous bone matrix has been demineralized to contain about 6% to about 15% by weight residual calcium.

29. The implant of claim 19, wherein the at least one demineralized cancellous bone matrix is formed from human bone.

30. The implant of claim 19, wherein the at least one demineralized cancellous bone matrix is formed from non-human bone.

31. The implant of claim 19, which further comprises at least one additional component.

32. The implant of claim 31, wherein the at least one additional component comprises bone marrow aspirate, blood, platelet rich plasma, platelet rich plasma matrix, stem cells, epithelial cells, fibroblasts, osteoclasts, osteoblasts, chemotactic factors, growth factors, a carrier, cortical bone, or combinations thereof.

33. The implant of claim 32, wherein the carrier comprises saline, phosphate buffered solution, sodium hyaluronate, hyaluronic acid, or combinations thereof.

34. The implant of claim 32, wherein the at least one additional component comprises stem cells comprising mesenchymal stem cells.

35. The implant of claim 19, wherein the at least one demineralized cancellous bone matrix includes less than or equal to about 10% by weight of cortical bone.

36. The implant of claim 19, wherein the at least one demineralized cancellous bone matrix includes less than or equal to about 5% by weight of growth factors, or less than or equal to about 5% by weight of connective tissue.

37. A method of treating bone having a void or defect in a patient in need thereof, the method comprising inserting into the void or defect at least one demineralized cancellous bone matrix, wherein the at least one demineralized cancellous bone matrix comprises a cancellous bone matrix that has been demineralized to contain about 5% to about 20% by weight residual calcium, and wherein the at least one demineralized cancellous bone matrix is rigid and has dimensions, wherein one of the dimensions is a length between about 0.5 mm to about 20 mm.

38. The method of claim 37, wherein the at least one demineralized cancellous bone matrix promotes bone growth in the void or defect.

39. The method of claim 37, wherein the void or defect is related to a trauma or a cancer.

40. The method of claim 37 wherein the at least one demineralized cancellous bone matrix has a length between about 2 mm to about 12 mm.

41. The method of claim 37 wherein the at least one demineralized cancellous bone matrix has a length between about 5 mm to about 10 mm.

42. The method of claim 37, wherein each of the dimensions of the demineralized cancellous bone matrix is individually between about 0.5 mm to about 20 mm.

43. The method of claim 37, wherein each of the dimensions of the demineralized cancellous bone matrix is individually between about 2 mm to about 12 mm.

44. The method of claim 37, wherein each of the dimensions of the demineralized cancellous bone matrix is individually between about 5 mm to about 10 mm.

45. The method of claim 42, wherein the size of the individual dimensions can vary along the dimensions.

46. The method of claim 37 wherein the at least one demineralized cancellous bone matrix has a cuboidal shape.

47. The method of claim 37 wherein at least one surface of the at least one demineralized cancellous bone matrix has an irregular polygonal shape.

48. The method of claim 37 wherein the cancellous bone matrix has been demineralized to contain about 6% to about 15% by weight residual calcium.

49. The method of claim 37 wherein the at least one demineralized cancellous bone matrix is formed from human bone.

50. The method of claim 37 wherein the at least one demineralized cancellous bone matrix is formed from non-human bone.

51. The method of claim 37, wherein the at least one demineralized cancellous bone matrix further comprises at least one additional component.

52. The method of claim 51 wherein the at least one additional component comprises bone marrow aspirate, blood, platelet rich plasma, platelet rich plasma matrix, stem cells, epithelial cells, fibroblasts, osteoclasts, osteoblasts, chemotactic factors, growth factors, a carrier, cortical bone, or combinations thereof.

53. The method of claim 52, wherein the carrier comprises saline, phosphate buffered solution, sodium hyaluronate, hyaluronic acid, or combinations thereof.

54. The method of claim 52 wherein the at least one additional component comprises stem cells comprising mesenchymal stem cells.

55. The method of claim 37 wherein the at least one demineralized cancellous bone matrix includes less than or equal to about 10% by weight of cortical bone.

56. The method of claim 37 wherein the at least one demineralized cancellous bone matrix includes less than or equal to about 5% by weight of growth factors, or less than or equal to about 5% by weight of connective tissue.

57. A method of treating a spinal disc in a patient in need thereof, the method comprising:
forming at least one cavity located between two adjacent vertebral bodies; and
inserting into the cavity at least one demineralized cancellous bone matrix, wherein the at least one demineralized cancellous bone matrix comprises a cancellous bone matrix that has been demineralized to contain about 5% to about 20% by weight residual calcium, and wherein the at least one demineralized cancellous bone matrix is rigid and has dimensions, wherein one of the dimensions is a length between about 0.5 mm to about 20 mM.

58. The method of claim 57, wherein the cavity is located within the spinal disc.

59. The method of claim 57, wherein the at least one demineralized cancellous bone matrix promotes bone growth in the cavity.

60. The method of claim 57, wherein the at least one demineralized cancellous bone matrix is used to create a fusion between the two adjacent vertebral bodies.

61. The method of claim 57, wherein the spinal disc is degenerated and the method is for treating the degenerated spinal disc.

62. The method of claim 57 wherein the at least one demineralized cancellous bone matrix has a length between about 2 mm to about 12 mm.

63. The method of claim 57 wherein the at least one demineralized cancellous bone matrix has a length between about 5 mm to about 10 mm.

64. The method of claim 57, wherein each of the dimensions of the demineralized cancellous bone matrix is individually between about 0.5 mm to about 20 mm.

65. The method of claim 57, wherein each of the dimensions of the demineralized cancellous bone matrix is individually between about 2 mm to about 12 mm.

66. The method of claim 57, wherein each of the dimensions of the demineralized cancellous bone matrix is individually between about 5 mm to about 10 mm.

67. The method of claim 64, wherein the size of the individual dimensions can vary along the dimensions.

68. The method of claim 57 wherein the at least one demineralized cancellous bone matrix has a cuboidal shape.

69. The method of claim 57 wherein at least one surface of the at least one demineralized cancellous bone matrix has an irregular polygonal shape.

70. The method of claim 57 wherein the cancellous bone matrix has been demineralized to contain about 6% to about 15% by weight residual calcium.

71. The method of claim 57 wherein the at least one demineralized cancellous bone matrix is formed from human bone.

72. The method of claim 57 wherein the at least one demineralized cancellous bone matrix is formed from non-human bone.

73. The method of claim 57, wherein the at least one demineralized cancellous bone matrix further comprises at least one additional component.

74. The method of claim 73, wherein the at least one additional component comprises bone marrow aspirate, blood, platelet rich plasma, platelet rich plasma matrix, stem cells, epithelial cells, fibroblasts, osteoclasts, osteoblasts, chemotactic factors, growth factors, a carrier, cortical bone, or combinations thereof.

75. The method of claim 74, wherein the carrier comprises saline, phosphate buffered solution, sodium hyaluronate, hyaluronic acid, or combinations thereof.

76. The method of claim 74 wherein the at least one additional component comprises stem cells comprising mesenchymal stem cells.

77. The method of claim 57 wherein the at least one demineralized cancellous bone matrix includes less than or equal to about 10% by weight of cortical bone.

78. The method of claim 57 wherein the at least one demineralized cancellous bone matrix includes less than or equal to about 5% by weight of growth factors, or less than or equal to about 5% by weight of connective tissue.

79. A method of making a demineralized cancellous bone matrix comprising demineralizing a cancellous bone matrix to contain about 5% to about 20% by weight residual calcium, wherein the demineralized cancellous bone matrix is rigid and has dimensions, wherein one of the dimensions is a length between about 0.5 mm to about 20 mm, and wherein the demineralized cancellous bone matrix is made by a process comprising:
a. cutting bone to obtain the cancellous bone matrix, wherein the cancellous bone matrix has a length between about 0.5 mm to about 20 mm; and
b. demineralizing the cancellous bone matrix so that the residual calcium in the cancellous bone matrix is about 5% to about 20% by weight.

80. The method of claim 79, wherein the cancellous bone matrix has a length between about 2 mm to about 12 mm.

81. The method of claim 79, wherein the cancellous bone matrix has a length between about 5 mm to about 10 mm.

82. The method claim 79, wherein each of the dimensions of the demineralized cancellous bone matrix is individually between about 0.5 mm to about 20 mm.

83. The method of claim 79, wherein each of the dimensions of the demineralized cancellous bone matrix is individually between about 2 mm to about 12 mm.

84. The method of claim 79, wherein each of the dimensions of the demineralized cancellous bone matrix is individually between about 5 mm to about 10 mm.

85. The method of claim 82, wherein the size of the individual dimensions can vary along the dimensions.

86. The method of claim 79, wherein the demineralized cancellous bone matrix has a cuboidal shape.

87. The method of claim 79, wherein the demineralized cancellous bone matrix has an irregular polygonal shape.

88. The method of claim 79, wherein the cancellous bone matrix is demineralized in hydrochloric acid.

89. The method of claim 88, wherein the hydrochloric acid is about 0.2N to about 1.0N.

90. The method of claim 88, wherein the hydrochloric acid is about 0.3N to about 0.6N.

91. The method of claim 79, wherein the cancellous bone matrix is demineralized for about 2 minutes to about 15 minutes.

92. The method of claim 79, wherein the cancellous bone matrix is demineralized for about 5 minutes to about 10 minutes.

93. The method of claim 88, wherein the hydrochloric acid is about 0.2N to about 0.5N and wherein the cancellous bone matrix is demineralized for about 5 minutes to about 10 minutes.

94. The method of claim 79, wherein the cancellous bone matrix is demineralized to contain about 6% to about 15% by weight residual calcium.

95. The method of claim 79, wherein the demineralized cancellous bone matrix is formed from human bone.

96. The method of claim 79, wherein the demineralized cancellous bone matrix is formed from non-human bone.

97. The method of claim 79, wherein the process further comprises exposing the demineralized cancellous bone matrix to one or more bone cleaning agents.

98. The method of claim 97, wherein the demineralized cancellous bone matrix is exposed to the one or more bone cleaning agents before demineralizing the cancellous bone matrix.

99. The method of claim 97, wherein the demineralized cancellous bone matrix is exposed to the one or more bone cleaning agents after demineralizing the cancellous bone matrix.

100. The method of claim 97, wherein the demineralized cancellous bone matrix is exposed to the one or more bone cleaning agents before and after demineralizing the cancellous bone matrix.

101. The method of claim 97, wherein the one or more bone cleaning agent comprises hydrogen peroxide, a detergent, an antibiotic, an alcohol, or combinations thereof.

102. The method of claim 97, wherein the demineralized cancellous bone matrix is sonicated in the one or more bone cleaning agent.

103. The method of claim 97, wherein the demineralized cancellous bone matrix is agitated in the one or more bone cleaning agent.

104. The method of claim 103, wherein the demineralized cancellous bone matrix is agitated by mechanical stirring.

105. The method of claim 97 wherein the demineralized cancellous bone matrix is soaked in the one or more bone cleaning agent.

106. The method of claim 79, wherein the process further comprises combining the demineralized cancellous bone matrix with at least one additional component.

107. The method of claim 106, wherein the at least one additional component comprises bone marrow aspirate, blood, platelet rich plasma, platelet rich plasma matrix, stem cells, epithelial cells, fibroblasts, osteoclasts, osteoblasts, chemotactic factors, growth factors, a carrier, cortical bone, or combinations thereof.

108. The method of claim 107, wherein the carrier comprises saline, phosphate buffered solution, sodium hyaluronate, hyaluronic acid, or combinations thereof.

109. The method of claim 107, wherein the at least one additional component comprises stem cells comprising mesenchymal stem cells.

110. The method of claim 79, wherein the demineralized cancellous bone matrix includes less than or equal to about 10% by weight of cortical bone.

111. The method of claim 79, wherein the demineralized cancellous bone matrix includes less than or equal to about 5% by weight of growth factors, or less than or equal to about 5% by weight of connective tissue.
